(19) United States
(12) Patent Application Publication
(10) Pub. No.: US 2024/0423985 A1
(43) Pub. Date: Dec. 26, 2024

(54) INHIBITORS OF HUMAN IMMUNODEFICIENCY VIRUS REPLICATION

(71) Applicants: VIIV HEALTHCARE UK (NO.5) LIMIT-ED, Middlesex (GB); VIIV HEALTHCARE UK (NO.5) LIMIT-ED, Stevenage (GB)

(72) Inventor: Gautam DALWADI, Collegeville, PA (US)

(21) Appl. No.: 18/700,402

(22) PCT Filed: Oct. 12, 2022
(86) PCT No.: PCT/IB2022/059780

§ 371 Date: Apr. 11, 2024

Related U.S. Application Data
(60) Provisional application No. 63/255,056, filed on Oct. 13, 2021.
(60) Provisional application No. 63/257,212, filed on Oct. 19, 2021.

(51) Int. Cl. A61K 31/519; A61K 9/08; A61K 9/10; A61K 31/517; A61K 47/10; A61K 47/12; A61K 47/24

(52) U.S. Cl. CPC A61K 31/519; A61K 9/08; A61K 9/10; A61K 31/517; A61K 47/10; A61K 47/12; A61K 47/24

(57) ABSTRACT

A pharmaceutical composition comprising the compound of Formula Ia, or Formula Ib, or a pharmaceutically acceptable salt thereof, is set forth:

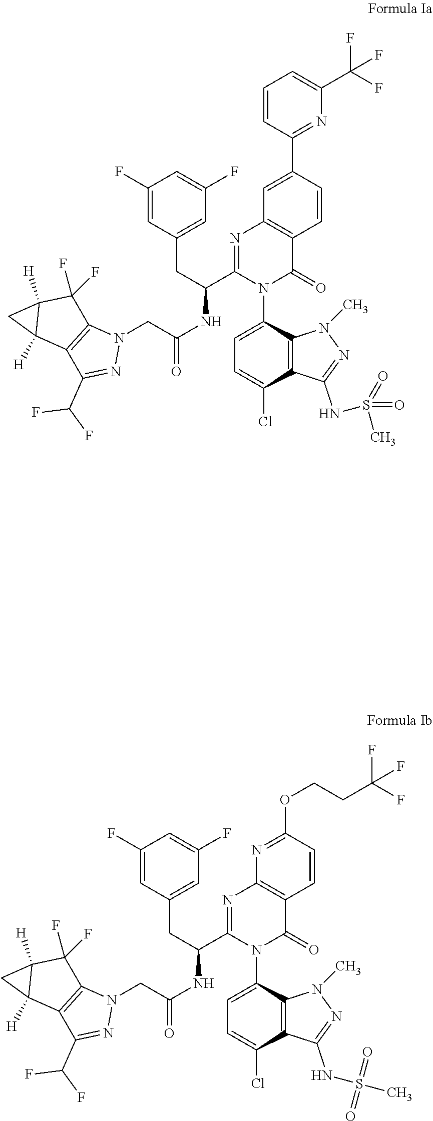

### FIELD OF THE INVENTION

The invention relates to compounds, pharmaceutical compositions, and methods for the treatment of human immunodeficiency virus (HIV) infection. More particularly, the invention provides pharmaceutical compositions containing inhibitors of HIV, and methods for using these compositions in the treatment of HIV infection.

### BACKGROUND OF THE INVENTION

Acquired immunodeficiency syndrome (AIDS) is the result of infection by HIV. HIV continues to be a major global public health issue. In 2015, an estimated 36.7 million people were living with HIV (including 1.8 million children)—a global HIV prevalence of 0.8%. The vast majority of this number live in low- and middle-income countries. In the same year, 1.1 million people died of AIDS-related illnesses.

Current therapy for HIV-infected individuals consists of a combination of approved anti-retroviral agents. Close to four dozen drugs are currently approved for HIV infection, either as single agents, fixed dose combinations or single tablet regimens; the latter two containing 2-4 approved agents. These agents belong to a number of different classes, targeting either a viral enzyme or the function of a viral protein during the virus replication cycle. Thus, agents are classified as either nucleotide reverse transcriptase inhibitors (NRTIs), non-nucleotide reverse transcriptase inhibitors (NNRTIs), protease inhibitors (PIS), integrase strand transfer inhibitors (INSTIs), or entry inhibitors (one, maraviroc, targets the host CCR5 protein, while the other, enfuvirtide, is a peptide that targets the gp41 region of the viral gp160 protein). In addition, a pharmacokinetic enhancer (cobicistat or ritonavir) can be used in combinations with antiretroviral agents (ARVs) that require boosting.

Certain potentially therapeutic compounds which appear to act by disrupting the normal functions of the HIV virus capsid have been described in the art. No currently approved drugs act by this mechanism and thus a compound acting through this mechanism would be a useful addition to the options available for the treatment of HIV infection.

WO 2020/084492 and WO 2020/254985 disclose certain Capsid Inhibitor compounds including the two compounds shown below which will be referred to in this application as the compounds of Formula Ia and Formula Ib.

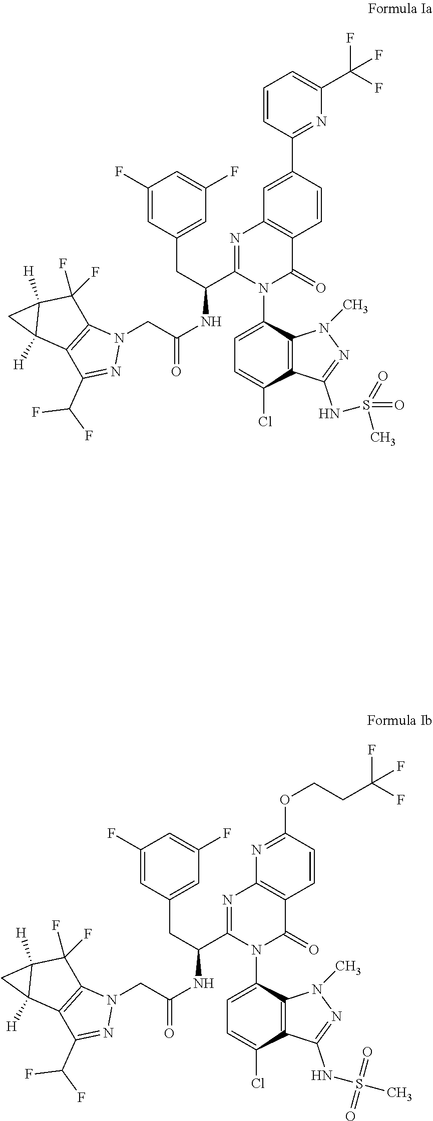

These compound provide advantages for pharmaceutical uses, for example, with regard to one or more of their mechanisms of action, binding, inhibition efficacy, target selectivity, solubility, safety profiles, bioavailability and/or reduced frequency of dosing. This disclosure teaches pharmaceutical compositions, methods of administration and methods of treatment utilizing these compounds.

### SUMMARY OF THE INVENTION

In one aspect, the present invention provides a pharmaceutical composition comprising a compound of Formula Ia or a pharmaceutically acceptable salt thereof,

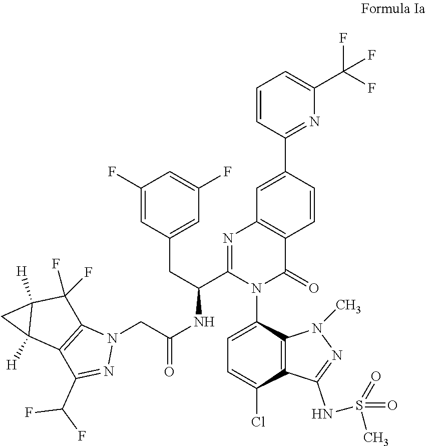

wherein the composition comprising polyethylene glycol (PEG) and ethanol.

In another aspect, the present invention provides a pharmaceutical composition comprising a compound of Formula Ia or a pharmaceutically acceptable salt thereof,

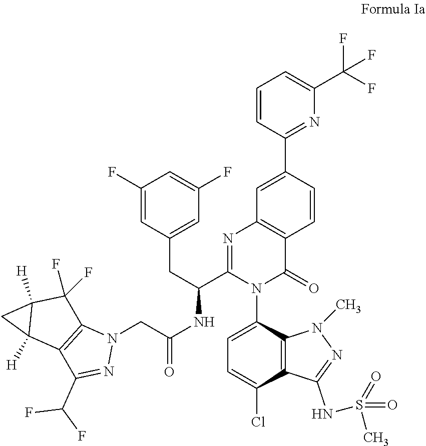

wherein the composition comprises water and contains less than 1% by weight of polyethylene glycol.

In still another aspect, the present invention provides a pharmaceutical composition comprising the compound of Formula Ib or a pharmaceutically acceptable salt thereof,

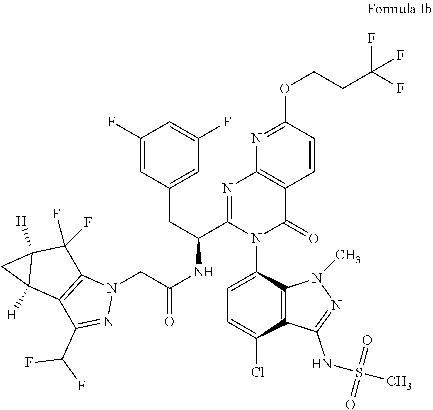

wherein the composition comprises polyethylene glycol (PEG) and ethanol.

In a further aspect, the invention provides a pharmaceutical composition comprising a compound of Formula Ia or a pharmaceutically acceptable salt thereof,

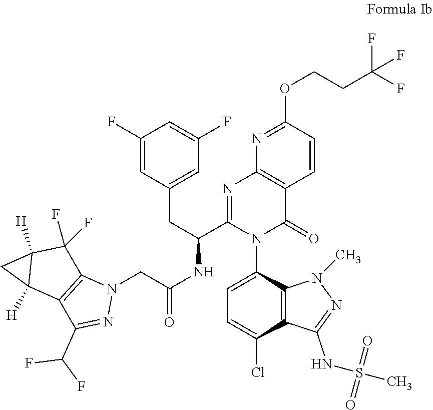

wherein the composition comprises water and contains less than 1% by weight of polyethylene glycol.

In another aspect, the present invention provides a method of treating HIV infection in a patient comprising administering a therapeutically effective amount of a pharmaceutical composition of the invention, as described below, to said patient.

In another aspect, the present invention provides a pharmaceutical composition of the invention, as described below, for use in therapy.

In another aspect, the present invention provides a pharmaceutical composition of the invention, as described below, for use in treating HIV infection in a patient.

In another aspect, the present invention provides the use of a pharmaceutical composition of the invention, as described below, in the manufacture of a medicament for the treatment of HIV infection in a patient.

### BRIEF DESCRIPTION OF THE FIGURES

FIGS. 1-3 summarize the results of PK experiments described below and summarized in Tables 1-3.

FIGS. 4-6 summarize the results of PK experiments described below and summarized in Tables 4-6.

FIGS. 7-9 summarize the results of PK experiments described below and summarized in Tables 7-9.

FIGS. 10-12 summarize the results of PK experiments described below and summarized in Tables 10-12.

FIGS. 13-14 summarize the results of PK experiments described below and summarized in Tables 13-14.

FIGS. 15-16 summarize the results of PK experiments described below and summarized in Tables 15-16.

FIGS. 17-18 summarize the results of PK experiments described below and summarized in Tables 17-18.

FIGS. 19-20 summarize the results of PK experiments described below and summarized in Tables 19-20.

FIGS. 21-22 summarize the results of PK experiments described below and summarized in Tables 21-22.

### DETAILED DESCRIPTION OF THE INVENTION

A compound of Formula Ia is known by the chemical name N—((S)-1-((3P)-3-(4-chloro-1-methyl-3-(methylsulfonamido)-1H-indazol-7-yl)-4-oxo-7-(6-(trifluoromethyl) pyridin-2-yl)-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. A method for making the compound of Formula Ia is found in published patent application WO 2020/084492.

A compound of Formula Ib is known by the chemical name N—((S)-1-(3-(4-chloro-1-methyl-3-(methylsulfonamido)-1H-indazol-7-yl)-4-oxo-7-(3,3,3-trifluoropropoxy)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. A method for making the compound of Formula Ib is found in published patent application WO 2020/254985.

Suitably, the compositions of the invention comprise a therapeutically effective amount of a compound of Formula Ia or a pharmaceutically acceptable salt thereof. In one embodiment, the compositions of the invention comprise a therapeutically effective amount of a compound of Formula Ia as a free base.

In one embodiment, the compositions of the invention comprise a therapeutically effective amount of a compound of Formula Ia which is amorphous.

Suitably, the compositions of the invention comprise a therapeutically effective amount of a compound of Formula Ib or a pharmaceutically acceptable salt thereof. In one embodiment, the compositions of the invention comprise a therapeutically effective amount of a compound of Formula Ib as a free base.

In one embodiment, the compositions of the invention comprise a therapeutically effective amount of a compound of Formula Ib which is amorphous.

As used herein, “therapeutically effective amount” in reference to a compound, its salt, or a pharmaceutical composition of the invention comprising said compound or its salt, or other pharmaceutically-active agent or composition, means an amount of the compound, its salt or a pharmaceutical composition of the invention comprising said compound or its salt, sufficient to treat the patient's condition but low enough to avoid serious side effects (at a reasonable benefit/risk ratio) within the scope of sound medical judgment. Thus, e.g., a therapeutically effective amount of a compound of Formula Ia or Formula Ib, or a pharmaceutically acceptable salt thereof, or a pharmaceutical composition comprising the compound of Formula Ia or the compound of Formula Ib, or its salt, in a quantity that, when administered to a patient in need thereof, is sufficient to modulate the activity of HIV capsid such that the disease condition which is mediated by that activity is treated, including reduced, alleviated, or prevented. A therapeutically effective amount of a compound, its salt or a pharmaceutical composition comprising the compound or its salt, will vary with the particular compound chosen (e.g., consider the potency, efficacy, and half-life of the compound); the route of administration chosen; the condition being treated; the severity of the condition being treated; the age, size, weight, and physical condition of the patient being treated; the medical history of the patient to be treated; the duration of the treatment; the nature of concurrent therapy; the desired therapeutic effect; and like factors, but can nevertheless be routinely determined by the skilled artisan.

In one aspect, a composition of this invention comprises polyethylene glycol and ethanol. It will be understood by the skilled artisan that the chemical formula for polyethylene glycol (PEG) can be generally written as H—(O—CH₂—CH₂)ₙ—OH. In one embodiment, the composition of the invention is a homogeneous solution.

In one embodiment, the invention provides a composition further comprising water. In another embodiment, the invention provides a composition further comprising lecithin. In yet another embodiment, the invention provides a composition further comprising propylene glycol. In still another embodiment, the invention provides a composition further comprising benzyl alcohol. In still yet another embodiment, the invention provides a composition further comprising benzyl benzoate. In another embodiment, the invention provides a composition further comprising sucrose acetate isobutyrate (SAIB). In yet another embodiment, the invention provides a composition further comprising sesame oil.

In still yet another embodiment, the invention provides a composition further comprising one or more components which are water, lecithin, propylene glycol, benzyl alcohol, benzyl benzoate, SAIB, or sesame oil. In one embodiment, the invention provides a composition further comprising one or more components which are water, lecithin, propylene glycol, benzyl alcohol, benzyl benzoate, or sesame oil. In one embodiment, the invention provides a composition further comprising one or more components which are water, lecithin, propylene glycol, benzyl alcohol, or sesame oil. In one embodiment, the invention provides a composition further comprising one or more components which are propylene glycol, benzyl alcohol, or sesame oil.

In one embodiment, the lecithin is egg-based. In another embodiment, the lecithin is soy-based and is about 80 weight % phosphatidylcholine or is about 100 weight % phosphatidylcholine.

In one aspect of the invention, the average molecular weight of polyethylene glycol is about 200 (PEG 200).

In another aspect of the invention, the average molecular weight of polyethylene glycol is about 300 (PEG 300).

In another aspect of the invention, the average molecular weight of polyethylene glycol is about 400 (PEG 400).

Suitably, the amount of a component present in the composition is expressed as a weight % relative to total mass of the formulation.

In one aspect of the invention, the amount of a compound of Formula Ia, or a pharmaceutically acceptable salt thereof, present in the composition expressed as weight %, is between about 5-50%. In one embodiment of the invention, the amount of a compound of Formula Ia, or a pharmaceutically acceptable salt thereof, present in the composition is between about 5-30%. In one embodiment of the invention, the amount of a compound of Formula Ia, or a pharmaceutically acceptable salt thereof, present in the composition is between about 5-35%. In one embodiment of the invention, the amount of a compound of Formula Ia, or a pharmaceutically acceptable salt thereof, present in the composition is about 10-25%. In one embodiment of the invention, the amount of a compound of Formula Ia, or a pharmaceutically acceptable salt thereof, present in the composition is about 10-30%. In one embodiment of the invention, the amount of a compound of Formula Ia, or a pharmaceutically acceptable salt thereof, present in the composition is between about 15-30%. In one embodiment of the invention, the amount of a compound of Formula Ia, or a pharmaceutically acceptable salt thereof, present in the composition is between about 20-30%. In one embodiment of the invention, the amount of a compound of Formula Ia, or a pharmaceutically acceptable salt thereof, present in the composition is between about 25-35%. In one embodiment of the invention, the amount of a compound of Formula Ia, or a pharmaceutically acceptable salt thereof, present in the composition is between about 30-40%. In one embodiment of the invention, the amount of a compound of Formula Ia, or a pharmaceutically acceptable salt thereof, present in the composition is between about 35-45%. In one embodiment of the invention, the amount of a compound of Formula Ia, or a pharmaceutically acceptable salt thereof, present in the composition is between about 40-50%.

In one aspect of the invention, the amount of a compound of Formula Ib, or a pharmaceutically acceptable salt thereof, present in the composition, expressed as weight %, is between about 5-50%.

In one embodiment of the invention, the amount of a compound of Formula Ib, or a pharmaceutically acceptable salt thereof, present in the composition is between about 5-30%. In one embodiment of the invention, the amount of a compound of Formula Ib, or a pharmaceutically acceptable salt thereof, present in the composition is between about 5-35%. In one embodiment of the invention, the amount of a compound of Formula Ib, or a pharmaceutically acceptable salt thereof, present in the composition is between about 10-25%. In one embodiment of the invention, the amount of a compound of Formula Ib, or a pharmaceutically acceptable salt thereof, present in the composition is between about 10-30%. In one embodiment of the invention, the amount of a compound of Formula Ib, or a pharmaceutically acceptable salt thereof, present in the composition is between about 15-30%. In one embodiment of the invention, the amount of a compound of Formula Ib, or a pharmaceutically acceptable salt thereof, present in the composition is between about 20-30%. In one embodiment of the invention, the amount of a compound of Formula Ib, or a pharmaceutically acceptable salt thereof, present in the composition is between about 25-35%. In one embodiment of the invention, the amount of a compound of Formula Ib, or a pharmaceutically acceptable salt thereof, present in the composition is between about 30-40%. In one embodiment of the invention, the amount of a compound of Formula Ib, or a pharmaceutically acceptable salt thereof, present in the composition is between about 35-45%. In one embodiment of the invention, the amount of a compound of Formula Ib, or a pharmaceutically acceptable salt thereof, present in the composition is between about 40-50%.

Suitably, the compositions of the invention are administered subcutaneously.

In one embodiment, the invention provides a homogeneous solution for subcutaneous administration. In one embodiment, the invention provides a homogeneous solution comprising an amorphous form of a compound of Formula Ia for subcutaneous administration. In one embodiment, the invention provides a homogeneous solution comprising an amorphous form of a compound of Formula Ib for subcutaneous administration.

In one embodiment, the invention provides a heterogeneous suspension for subcutaneous administration. In one embodiment, the invention provides a heterogeneous suspension comprising an amorphous form of a compound of Formula Ia for subcutaneous administration. In one embodiment, the invention provides a heterogeneous suspension comprising an amorphous form of a compound of Formula Ib for subcutaneous administration.

Suitably, the compositions of the invention are administered intramuscularly.

Suitably, the compositions of the invention are administered intravenously.

The compositions of the invention comprise a vehicle or carrier, which is an inert medium used as a solvent or diluent in which the active agent, Formula Ia or Formula Ib, is formulated or administered. Suitable vehicles for the compositions of this invention include, but are not limited to, ethanol (up to about 35 weight %), polyethylene glycol (up to about 85 weight %), modified polyethylene glycol (up to about 85 weight %), propylene glycol (up to about 60 weight %), N-Methyl-2-pyrrolidone (NMP) (up to about weight %), Dimethylacetamide (DMA) (up to about 50%), dimethylsulfoxide (DMSO) (up to about 5 weight %), water, ethyl lactate, dimethyl isosorbide, and the like. It will be understood that one or more solvents may comprise the vehicle for a particular pharmaceutical composition.

The compositions of the invention optionally comprise an oil. Suitable oils for the compositions of this invention include, but are not limited to, sesame oil, soybean oil, castor oil, medium chain triglyceride, safflower oil, and the like. Suitably, for an emulsion the oil is present in an amount of from about 0 to about 50 weight %. Suitably, for an oily solution, the oil is present in an amount of up to about 100 weight %. In one embodiment, the invention provides a homogeneous solution comprising an oil.

The compositions of the invention optionally comprise a surfactant. Suitable surfactants include, but are not limited to, a phospholipid (up to about 25 weight %), a poloxamer (up to about 7 weight %), a polysorbate (up to about 7 weight %), a sorbitan ester (aka spans) (up to about 7 weight %), and the like. In one embodiment, the invention provides a composition comprising a phospholipid surfactant. In one embodiment, the invention provides a composition comprising a phospholipid surfactant which is lecithin. In one embodiment, the invention provides a composition comprising Poloxamer 338. In one embodiment, the invention provides a composition comprising Poloxamer 188. In one embodiment, the invention provides a composition comprising Poloxamer 338 or Poloxamer 188.

In one embodiment, the invention provides a heterogeneous suspension comprising a surfactant. In one embodiment, the invention provides a heterogeneous suspension comprising a surfactant. In one embodiment, the invention provides a heterogeneous suspension comprising lecithin. In one embodiment, the invention provides a heterogeneous suspension comprising Poloxamer 338. In one embodiment, the invention provides a heterogeneous suspension comprising Poloxamer 188. In one embodiment, the invention provides a heterogeneous suspension comprising Poloxamer 338 or Poloxamer 188.

If the composition of the invention is a heterogeneous suspension, it optionally comprises an iso-osmolarity/tonicity agent. Suitable iso-osmolarity/tonicity agents include but are not limited to, mannitol (about 1 to about 5 weight %), trehalose (about 7 to about 10 weight %), sucrose (about 7 to about 10 weight %), glucose (about 3 to about 5 weight %), dextrose (about 3 to about 5 weight %), sodium chloride (about 0.45 to about 0.9 weight %), potassium chloride (about 0.45 to about 0.9 weight %), and the like. In one embodiment, the invention provides a heterogeneous suspension comprising mannitol.

The compositions of the invention optionally comprise a buffering agent. Suitable buffering agents for the compositions of the invention include, but are not limited to, acetate, citrate, tartrate, malic acid and its salt, NaOH and HCl, format histidine, phosphate, TRIS, borate, and the like. In one embodiment, the invention provides a composition comprising a buffering agent in the amount of about 1 mM to about 20 mM.

In one aspect, the invention provides a composition which is a micro-suspension. In one embodiment, the invention provides a micro-suspension composition comprising a viscosity modifying agent. Suitable viscosity modifying agents for the compositions of the invention include, but are not limited to, sodium carboxymethyl cellulose, hyaluronic acids, PVP-K-12, K-19, hydroxy ethyl starch, and the like. In one embodiment, the invention provides a composition comprising levels of viscosity modifying agents from 0 to about 1 weight %. In another embodiment, the invention provides a micro-suspension composition comprising a bulking agent. Suitable bulking agents for the compositions of the invention include, but are not limited to, mannitol (about 3 to about 5 weight %), trehalose (about 7 to about 10 weight %), sucrose (about 7 to about 10 weight %), glucose (about 3 to about 5 weight %), dextrose (about 3 to about 5 weight %), and the like. In one embodiment, the invention provides a composition which is a lyophilized micro-suspension.

In another aspect, the invention provides a pharmaceutical composition wherein the amount of polyethylene glycol present in the composition, expressed as weight %, is between about 10-55%. In one embodiment of the invention, the amount of polyethylene glycol present in the composition is between about 15-50%. In a second embodiment of the invention, the amount of polyethylene glycol present in the composition is between about 20-50%. In one embodiment of the invention, the amount of polyethylene glycol present in the composition is between about 20-40%. In one embodiment of the invention, the amount of polyethylene glycol present in the composition is between about 30-50%. In one embodiment of the invention, the amount of polyethylene glycol present in the composition is between about 40-50%.

In another aspect, the invention provides a pharmaceutical composition wherein the amount of ethanol present in the composition, expressed as weight %, is between about 1-35%. In one embodiment of the invention, the amount of ethanol present in the composition is between about 5-30%. In one embodiment of the invention, the amount of ethanol present in the composition is between about 5-25%. In one embodiment of the invention, the amount of ethanol present in the composition is between about 10-30%. In one embodiment of the invention, the amount of ethanol present in the composition is between about 10-25%. In one embodiment of the invention, the amount of ethanol present in the composition is between about 15-30%. In one embodiment of the invention, the amount of ethanol present in the composition is between about 15-20%. In one embodiment of the invention, the amount of ethanol present in the composition is between about 15-25%. In one embodiment of the invention, the amount of ethanol present in the composition is between about 20-25%. In one embodiment of the invention, the amount of ethanol present in the composition is between about 25-35%.

Suitably, the pharmaceutical composition comprises mannitol. In another aspect, the invention provides a pharmaceutical composition wherein the amount of mannitol is present in the composition expressed as weight % is between 1-5%. In one embodiment of the invention, the amount of mannitol present in the composition is between about 2-4%.

Suitably, the pharmaceutical composition comprises lecithin. In another aspect, the invention provides a pharmaceutical composition wherein the amount of lecithin present in the composition expressed as weight % is between about 1-25%. In one embodiment of the invention, the amount of lecithin in the composition is between about 5-25%. In one embodiment of the invention, the amount of lecithin in the composition is between about 10-20%. In one embodiment of the invention, the amount of lecithin in the composition is between about 1-5%. In another embodiment of the invention, the amount of lecithin present in the composition is about 1%, 2%, 3%, 4%, or 5%.

In one embodiment of the invention, the lecithin is egg-based. In another embodiment of the invention, the lecithin is soy-based. In one embodiment, if soy-based, the lecithin is about 80 weight % phosphatidylcholine. In one embodiment, if soy-based, the lecithin is 100 weight % phosphatidylcholine.

In another aspect, the invention provides a composition which is a homogeneous solution.

In yet another aspect, the invention provides a composition which is a heterogeneous suspension.

In one aspect, the invention provides a pharmaceutical composition wherein the amount of water present in the composition, as measured by Karl Fischer titration, is about 1%, 2%, 3%, 4%, or 5%. In one embodiment of the invention, the amount of water present in the composition is less than about 3%. In one embodiment of the invention, the amount of water present in the composition is less than about 2.5%. In one embodiment of the invention, the amount of water present in the composition is less than about 2%. In one embodiment of the invention, the amount of water present in the composition is less than about 1.5%. In one embodiment of the invention, the amount of water in the composition is less than about 1%.

It will be understood that all the above embodiments apply to compositions of the invention comprising Formula Ia, or a pharmaceutically acceptable salt thereof. It will be understood that all the above embodiments apply to compositions of the invention comprising Formula Ib, or a pharmaceutically acceptable salt thereof. It will be understood that the above embodiments apply to compositions of the invention comprising a compound of Formula Ia as a free base. It will be understood that the above embodiments apply to compositions of the invention comprising a compound of Formula Ia as a free base. It will be understood that the above embodiments apply to compositions of the invention comprising a compound of Formula Ia as an amorphous compound, either as a pharmaceutically acceptable salt thereof, or as a free base. It will be understood that the above embodiments apply to compositions of the invention comprising a compound of Formula Ib as an amorphous compound, either as a pharmaceutically acceptable salt thereof, or as a free base.

In another aspect, the invention provides a pharmaceutical composition comprising about 20% by weight of a compound of Formula Ia or a compound of Formula Ib, about 45% by weight of PEG200, about 20% by weight of ethanol, and about 15% by weight of lecithin. In another aspect, the invention provides a pharmaceutical composition comprising about 30% by weight of a compound of Formula Ia or a compound of Formula Ib, about 45% by weight of PEG200, and about 25% by weight of ethanol. In another aspect, the invention provides a composition comprising about 30% by weight of a compound of Formula Ia or a compound of Formula Ib, about 50% by weight of PEG200, and about 20% by weight of ethanol. In another aspect, the invention provides a composition comprising about 20% by weight of a compound of Formula Ia or a compound of Formula Ib, about 55% by weight of PEG200, and about 20% by weight of ethanol. In yet another aspect, the invention provides a composition comprising about 19% by weight of a compound of Formula Ia, about 61% by weight of PEG200 and about 20% by weight of ethanol.

### Heterogeneous Suspension

In one aspect, the composition of the invention comprises water and contains less than 1% by weight of polyethylene glycol. In another aspect, the composition of the invention is a heterogeneous suspension.

In one aspect, the invention provides a composition in which the suspended solids comprise a compound of Formula Ia, or a pharmaceutically acceptable salt thereof. In another aspect, the invention provides a composition in which the suspended solids comprise a compound of Formula Ib, or a pharmaceutically acceptable salt thereof. In one aspect, the invention provides a composition in which the suspended solids comprise a compound of Formula Ia, as the free base. In one aspect, the invention provides a composition in which the suspended solids comprise a compound of Formula Ib, as the free base. It will be understood that the above embodiments apply to compositions of the invention in which the suspended solids comprise a compound of Formula Ia as an amorphous compound, either as a pharmaceutically acceptable salt thereof, or as a free base. It will be understood that the above embodiments apply to compositions of the invention in which the suspended solids comprise a compound of Formula Ib as an amorphous compound, either as a pharmaceutically acceptable salt thereof, or as a free base.

In one embodiment of the invention, the composition of the solids which are suspended is about 20% by weight of a compound of Formula Ia. In another embodiment of the invention, the composition of the solids which are suspended is about 25% by weight of a compound of Formula Ia. In another embodiment of the invention, the composition of the solids which are suspended is about 30% by weight of a compound of Formula Ia. In another embodiment of the invention, the composition of the solids which are suspended is about 35% by weight of a compound of Formula Ia. In another embodiment of the invention, the composition of the solids which are suspended is about 40% by weight of a compound of Formula Ia. In another embodiment of the invention, the composition of the solids which are suspended is about 45% by weight of a compound of Formula Ia. In another embodiment of the invention, the composition of the solids which are suspended is about 50% by weight of a compound of Formula Ia. It will be understood that these embodiments apply to compositions of the invention in which the suspended solids comprise a compound of Formula Ia as a pharmaceutically acceptable salt or as a free base, or as an amorphous compound, either as a pharmaceutically acceptable salt thereof, or as a free base.

In one embodiment of the invention, the composition of the solids which are suspended is about 20% by weight of a compound of Formula Ib. In another embodiment of the invention, the composition of the solids which are suspended is about 25% by weight of a compound of Formula Ib. In another embodiment of the invention, the composition of the solids which are suspended is about 30% by weight of a compound of Formula Ib. In another embodiment of the invention, the composition of the solids which are suspended is about 35% by weight of a compound of Formula Ia. In another embodiment of the invention, the composition of the solids which are suspended is about 40% by weight of a compound of Formula Ib. In another embodiment of the invention, the composition of the solids which are suspended is about 45% by weight of a compound of Formula Ib. In another embodiment of the invention, the composition of the solids which are suspended is about 50% by weight of a compound of Formula Ib. It will be understood that these embodiments apply to compositions of the invention in which the suspended solids comprise a compound of Formula Ib as a pharmaceutically acceptable salt or as a free base, or as an amorphous compound, either as a pharmaceutically acceptable salt thereof, or as a free base.

In one aspect, the invention provides a composition further comprising one or more of the following excipients, sodium acetate, acetic acid, mannitol, sodium chloride, Poloxamer 338, or Poloxamer 188. In one embodiment, the invention provides a pharmaceutical composition comprising Poloxamer 338 or Poloxamer 188. In one embodiment, the invention provides a pharmaceutical composition comprising Poloxamer 338 and Poloxamer 188. In one embodiment, the invention provides a pharmaceutical composition comprising mannitol or sodium chloride. In one embodiment, the invention provides a pharmaceutical composition comprising mannitol and sodium chloride. In one embodiment, the invention provides a pharmaceutical composition comprising sodium acetate or acetic acid. In one embodiment, the invention provides a pharmaceutical composition comprising sodium acetate and acetic acid.

Suitably, the mass of a compound of Formula Ia or a compound of Formula Ib is expressed relative to the total volume of the formulation. In one embodiment, the composition comprises a compound of Formula Ia or a compound of Formula Ib at a concentration between about 50-500 mg/mL. In another embodiment, the composition comprises a compound of Formula Ia or a compound of Formula Ib at a concentration between about 150-300 mg/mL. In yet another embodiment, the composition comprises a compound of Formula Ia or a compound of Formula Ib at a concentration between about 200-300 mg/mL. In still another embodiment, the composition comprises a compound of Formula Ia or a compound of Formula Ib at a concentration between about 250-350 mg/mL. In still yet another embodiment, the composition comprises a compound of Formula Ia or a compound of Formula Ib at a concentration between about 300-400 mg/mL. In a further embodiment, the composition comprises a compound of Formula Ia or a compound of Formula Ib at a concentration between about 350-450 mg/mL. In another embodiment, the composition comprises a compound of Formula Ia or a compound of Formula Ib at a concentration between about 400-500 mg/mL. In one aspect, the composition comprises a compound of Formula Ia or a compound of Formula Ib at a concentration of about 200 mg/mL, about 225 mg/mL, about 250 mg/mL, about 275 mg/mL, about 300 mg/mL, about 325 mg/mL, about 350 mg/mL, about 375 mg/mL, about 400 mg/mL, about 450 mg/ml or about 500 mg/mL. In one embodiment, the invention provides a pharmaceutical composition comprising a compound of Formula Ia or a compound of Formula Ib at a concentration of about

In one aspect, the invention provides a composition comprising about 300 mg/ml of a compound of Formula Ia or a compound of Formula Ib, about 5.4% by weight of P338, about 3.5% by weight of mannitol, and the remainder of the formulation as water or aqueous acetate buffer.

In one aspect the compositions of this invention further comprise one or more of glycerol, polyvinylpyrrolidone K19, polyvinylpyrrolidone K12, Span, urea, NMP, ethyl lactate, polysorbate 80, or Polysorbate 20.

In one aspect, the pharmaceutical composition of this invention comprises a therapeutically effective amount of the compound of Formula Ia, or a pharmaceutically acceptable salt thereof.

In one aspect, the pharmaceutical composition of this invention comprises a therapeutically effective amount of the compound of Formula Ib, or a pharmaceutically acceptable salt thereof.

In one embodiment, the pharmaceutical composition of this invention comprises about 20%-30% by weight of a compound of Formula Ia, or a pharmaceutically acceptable salt thereof. In one embodiment, the pharmaceutical composition of this invention comprises about 20%-30% by weight of a compound of Formula Ia as the free base. In another embodiment, the pharmaceutical composition of the invention comprises about 20%-30% by weight of a compound of Formula Ib, or a pharmaceutically acceptable salt thereof. In one embodiment, the pharmaceutical composition of this invention comprises about 20%-30% by weight of a compound of Formula Ib as the free base.

In one embodiment, the pharmaceutical composition of this invention comprises about 20% by weight of a compound of Formula Ia, or a pharmaceutically acceptable salt thereof. In one embodiment, the pharmaceutical composition of this invention comprises about 20% by weight of a compound of Formula Ia as the free base. In another embodiment, the pharmaceutical composition of the invention comprises about 20% by weight of a compound of Formula Ib, or a pharmaceutically acceptable salt thereof. In one embodiment, the pharmaceutical composition of this invention comprises about 20% by weight of a compound of Formula Ib as the free base.

In one embodiment, the pharmaceutical composition of this invention comprises about 30% by weight of a compound of Formula Ia, or a pharmaceutically acceptable salt thereof. In one embodiment, the pharmaceutical composition of this invention comprises about 30% by weight of a compound of Formula Ia as the free base. In another embodiment, the pharmaceutical composition of the invention comprises about 30% by weight of a compound of Formula Ib, or a pharmaceutically acceptable salt thereof. In one embodiment, the pharmaceutical composition of this invention comprises about 30% by weight of a compound of Formula Ib as the free base.

It will be understood that the above embodiments apply to compositions of the invention comprising a compound of Formula Ia as an amorphous compound, either as a pharmaceutically acceptable salt thereof, or as a free base. It will be understood that the above embodiments apply to compositions of the invention comprising a compound of Formula Ib as an amorphous compound, either as a pharmaceutically acceptable salt thereof, or as a free base.

Suitably, in one aspect, the particle diameter of a compound of Formula Ia or a compound of Formula Ib is measured with a laser diffraction technique. This type of analysis is used in general practice for particle size characterization. An example of equipment capable of performing this analysis is a Malvern Mastersizer MS3000 instrument. Particle sizes are reported as percentiles of a distribution. Percentiles (e.g. X50) refer to the percent volume out of the total volume of the material tested which has an equivalent spherical diameter less than the reported value. The term “mean particle diameter” refers to X50 which is interchangeable with D50 or the 50th percentile distribution.

In one embodiment, the mean particle diameter of a compound of Formula Ia or a compound of Formula Ib is ≤ about 0.2 μm. In another embodiment, the mean particle diameter of a compound of Formula Ia or a compound of Formula Ib ranges between about 0.2 μm to about 0.5 μm. In another embodiment, the mean particle diameter of a compound of Formula Ia or a compound of Formula Ib ranges between about 0.5 μm to about 3 μm. In another embodiment, the mean particle diameter of a compound of Formula Ia or a compound of Formula Ib ranges between about 3 μm to about 5 μm. In another embodiment, the mean particle diameter of a compound of Formula Ia or a compound of Formula Ib ranges between about 5 μm to about 10 μm.

In one embodiment of the invention, for a compound of Formula Ia, the D10 is <0.9 μM, the D50 is <2 μM and the D90 is <4 μM. In one embodiment of the invention, for a compound of Formula Ib, the D10 is <0.9 μM, the D50 is <2 μM and the D90 is <4 μM.

In one aspect, instead of the specific stereoisomers depicted above in Formula Ia and Formula Ib, the composition of the invention comprises any of the isomers of a compound of Formula Ia or a compound of Formula Ib and they are included in the scope of this invention.

In one aspect the depicted stereoisomers in Formulas Ia and Ib are ≥95% of all stereoisomers of the same chemical formula.

The salts of the invention are pharmaceutically acceptable. Such salts may be acid addition salts or base addition salts. For a review of suitable pharmaceutically acceptable salts see, for example, Berge et al, J. Pharm, Sci., 66, 1-19, 1977.

Representative pharmaceutically acceptable acid addition salts include, but are not limited to, 4-acetamidobenzoate, acetate, adipate, alginate, ascorbate, aspartate, benzenesulfonate (besylate), benzoate, bisulfate, bitartrate, butyrate, calcium edetate, camphorate, camphorsulfonate (camsylate), caprate (decanoate), caproate (hexanoate), caprylate (octanoate), cinnamate, citrate, cyclamate, digluconate, 2,5-dihydroxybenzoate, disuccinate, dodecylsulfate (estolate), edetate (ethylenediaminetetraacetate), estolate (lauryl sulfate), ethane-1,2-disulfonate (edisylate), ethanesulfonate (esylate), formate, fumarate, galactarate (mucate), gentisate (2,5-dihydroxybenzoate), glucoheptonate (gluceptate), gluconate, glucuronate, glutamate, glutarate, glycerophosphorate, glycolate, hexylresorcinate, hippurate, hydrabamine (N,N′-di(dehydroabietyl)-ethylenediamine), hydrobromide, hydrochloride, hydroiodide, hydroxynaphthoate, isobutyrate, lactate, lactobionate, laurate, malate, maleate, malonate, mandelate, methanesulfonate (mesylate), methylsulfate, mucate, naphthalene-1,5-disulfonate (napadisylate), naphthalene-2-sulfonate (napsylate), nicotinate, nitrate, oleate, palmitate, p-aminobenzenesulfonate, p-aminosalicyclate, pamoate (embonate), pantothenate, pectinate, persulfate, phenylacetate, phenylethylbarbiturate, phosphate, polygalacturonate, propionate, p-toluenesulfonate (tosylate), pyroglutamate, pyruvate, salicylate, sebacate, stearate, subacetate, succinate, sulfamate, sulfate, tannate, tartrate, teoclate (8-chlorotheophyllinate), thiocyanate, triethiodide, undecanoate, undecylenate, and valerate.

Representative pharmaceutically acceptable base addition salts include, but are not limited to, aluminium, 2-amino-2-(hydroxymethyl)-1,3-propanediol (TRIS, tromethamine), arginine, benethamine (N-benzylphenethylamine), benzathine (N,N′-dibenzylethylenediamine), bis-(2-hydroxyethyl)amine, bismuth, calcium, chloroprocaine, choline, clemizole (1-p chlorobenzyl-2-pyrrolildine-1′-ylmethylbenzimidazole), cyclohexylamine, dibenzylethylenediamine, diethylamine, diethyltriamine, dimethylamine, dimethylethanolamine, dopamine, ethanolamine, ethylenediamine, L-histidine, iron, isoquinoline, lepidine, lithium, lysine, magnesium, meglumine (N-methylglucamine), piperazine, piperidine, potassium, procaine, quinine, quinoline, sodium, strontium, t-butylamine, and zinc.

In one embodiment, the salt of a compound of Formula Ia is a sodium salt. In another embodiment, the salt of a compound of Formula Ib is a sodium salt. In another embodiment, the salt of a compound of Formula Ia is a potassium salt. In another embodiment, the salt of a compound of Formula Ib is a potassium salt.

In another aspect the present invention discloses methods of preventing HIV infection in a patient or reducing the risk of infection, comprising administering a pharmaceutical composition of the invention. Pre-exposure prophylaxis (or PrEP) is when people at risk for HIV infection take HIV antiretroviral medicine to lower their chances of acquiring HIV infection. PrEP has been shown to be effective in reducing the risk of infection. As used herein, “HIV” or “Human Immunodeficiency Virus” refers to HIV-1 and/or HIV-2. As used herein, “patient” refers to a human.

The compounds, salts and compositions of this invention are believed to have as their biological target the HIV capsid and thus their mechanism of action is to modify in one or more ways the function of the HIV capsid.

The compound of Formula Ia and Formula Ib and salts thereof, may be employed alone or in combination with other therapeutic agents, or a prodrug thereof. Combination therapies according to the present invention thus comprise the administration of at least one compound or a pharmaceutically acceptable salt thereof, of the invention, and the administration of at least one other agent which may be useful in the treatment of HIV infection. A compound or a pharmaceutically acceptable salt thereof, of the present invention, and the other agent may be formulated and administered together in a single pharmaceutical composition or may be formulated and administered separately. When formulated and administered separately, administration may occur simultaneously or sequentially in any order.

Suitable other agents are selected from the group consisting of, abacavir, atazanavir, bictegravir, cabotegravir, darunavir, delavirdine, didanosine, dideoxyinosine, dolutegravir, doravirine, efavirenz, elvitegravir, emtricitabine, etavirine, fosamprenavir, fostemsavir, GSK3640254, GSK3739937/VH3739937, indinavir, islatravir, lamivudine, lopinavir, maraviroc, N6LS, nelfinavir, nevirapine, raltegravir, rilpiverine, ritonavir, S-648414, saquinavir, stavudine, tipranavir, tenofovir, tenofovir alafenamide, tenofovir disoproxil fumarate, zalcitabine, zidovudine, and S-365598. In one embodiment, the invention provides a pharmaceutical composition comprising a compound of Formula Ia, or pharmaceutically acceptable salt thereof, and another therapeutic agent selected from the group consisting of abacavir, atazanavir, bictegravir, cabotegravir, darunavir, delavirdine, didanosine, dideoxyinosine, dolutegravir, doravirine, efavirenz, elvitegravir, emtricitabine, etavirine, fosamprenavir, fostemsavir, GSK3640254, GSK3739937/VH3739937, indinavir, islatravir, lamivudine, lopinavir, maraviroc, N6LS, nelfinavir, nevirapine, raltegravir, rilpiverine, ritonavir, S-648414, saquinavir, stavudine, tipranavir, tenofovir, tenofovir alafenamide, tenofovir disoproxil fumarate, zalcitabine, zidovudine, and S-365598. In another embodiment, the invention provides a pharmaceutical composition comprising a compound of Formula Ib, or pharmaceutically acceptable salt thereof, and another therapeutic agent selected from the group consisting of abacavir, atazanavir, bictegravir, cabotegravir, darunavir, delavirdine, didanosine, dideoxyinosine, dolutegravir, doravirine, efavirenz, elvitegravir, emtricitabine, etavirine, fosamprenavir, fostemsavir, GSK3640254, GSK3739937/VH3739937, indinavir, islatravir, lamivudine, lopinavir, maraviroc, N6LS, nelfinavir, nevirapine, raltegravir, rilpiverine, ritonavir, S-648414, saquinavir, stavudine, tipranavir, tenofovir, tenofovir alafenamide, tenofovir disoproxil fumarate, zalcitabine, zidovudine, and S-365598.

In one embodiment, the invention provides a therapeutically effective pharmaceutical composition comprising a compound of Formula Ia, and another therapeutic agent selected from the group consisting of abacavir, atazanavir, bictegravir, cabotegravir, darunavir, delavirdine, didanosine, dideoxyinosine, dolutegravir, doravirine, efavirenz, elvitegravir, emtricitabine, etavirine, fosamprenavir, fostemsavir, GSK3640254, GSK3739937/VH3739937, indinavir, islatravir, lamivudine, lopinavir, maraviroc, N6LS, nelfinavir, nevirapine, raltegravir, rilpiverine, ritonavir, S-648414, saquinavir, stavudine, tipranavir, tenofovir, tenofovir alafenamide, tenofovir disoproxil fumarate, zalcitabine, zidovudine, and S-365598. In another embodiment, the invention provides a pharmaceutical composition comprising a compound of Formula Ib and another therapeutic agent selected from the group consisting of abacavir, atazanavir, bictegravir, cabotegravir, darunavir, delavirdine, didanosine, dideoxyinosine, dolutegravir, doravirine, efavirenz, elvitegravir, emtricitabine, etavirine, fosamprenavir, fostemsavir, GSK3640254, GSK3739937/VH3739937, indinavir, islatravir, lamivudine, lopinavir, maraviroc, N6LS, nelfinavir, nevirapine, raltegravir, rilpiverine, ritonavir, S-648414, saquinavir, stavudine, tipranavir, tenofovir, tenofovir alafenamide, tenofovir disoproxil fumarate, zalcitabine, zidovudine, and S-365598.

In one embodiment, the invention provides a pharmaceutical composition comprising a compound of Formula Ia which is amorphous, and another therapeutic agent selected from the group consisting of abacavir, atazanavir, bictegravir, cabotegravir, darunavir, delavirdine, didanosine, dideoxyinosine, dolutegravir, doravirine, efavirenz, elvitegravir, emtricitabine, etavirine, fosamprenavir, fostemsavir, GSK3640254, GSK3739937/VH3739937, indinavir, islatravir, lamivudine, lopinavir, maraviroc, N6LS, nelfinavir, nevirapine, raltegravir, rilpiverine, ritonavir, S-648414, saquinavir, stavudine, tipranavir, tenofovir, tenofovir alafenamide, tenofovir disoproxil fumarate, zalcitabine, zidovudine, and S-365598. In another embodiment, the invention provides a pharmaceutical composition comprising a compound of Formula Ib which is amorphous, and another therapeutic agent selected from the group consisting of abacavir, atazanavir, bictegravir, cabotegravir, darunavir, delavirdine, didanosine, dideoxyinosine, dolutegravir, doravirine, efavirenz, elvitegravir, emtricitabine, etavirine, fosamprenavir, fostemsavir, GSK3640254, GSK3739937/VH3739937, indinavir, islatravir, lamivudine, lopinavir, maraviroc, N6LS, nelfinavir, nevirapine, raltegravir, rilpiverine, ritonavir, S-648414, saquinavir, stavudine, tipranavir, tenofovir, tenofovir alafenamide, tenofovir disoproxil fumarate, zalcitabine, zidovudine, and S-365598.

In one embodiment, the other agent is selected from the group consisting of abacavir, atazanavir, bictegravir, cabotegravir, dolutegravir, fostemsavir, lamivudine, maraviroc, rilpiverine, tenofovir disoproxil, tenofovir, tenofovir afenamide, islatravir, doravirine, preziata, S-648414, GSK3640254, N6LS, GSK3739937/VH3739937, and S-365598.

In one embodiment, the invention provides a pharmaceutical composition comprising a compound of Formula Ia, or a pharmaceutically acceptable salt thereof, and another therapeutic agent selected from the group consisting of abacavir, atazanavir, bictegravir, cabotegravir, dolutegravir, fostemsavir, lamivudine, maraviroc, rilpiverine, tenofovir disoproxil, tenofovir, tenofovir afenamide, islatravir, doravirine, preziata, S-648414, GSK3640254, N6LS, GSK3739937/VH3739937, and S-365598. In one embodiment, the invention provides a pharmaceutical composition comprising a compound of Formula Ib, or a pharmaceutically acceptable salt thereof, and another therapeutic agent selected from the group consisting of abacavir, atazanavir, bictegravir, cabotegravir, dolutegravir, fostemsavir, lamivudine, maraviroc, rilpiverine, tenofovir disoproxil, tenofovir, tenofovir afenamide, islatravir, doravirine, preziata, S-648414, GSK3640254, N6LS, GSK3739937/VH3739937, and S-365598.

In one embodiment, the invention provides a pharmaceutical composition comprising a compound of Formula Ia and another therapeutic agent selected from the group consisting of abacavir, atazanavir, bictegravir, cabotegravir, dolutegravir, fostemsavir, lamivudine, maraviroc, rilpiverine, tenofovir disoproxil, tenofovir, tenofovir afenamide, islatravir, doravirine, preziata, S-648414, GSK3640254, N6LS, GSK3739937/VH3739937, and S-365598. In one embodiment, the invention provides a pharmaceutical composition comprising a compound of Formula Ib and another therapeutic agent selected from the group consisting of abacavir, atazanavir, bictegravir, cabotegravir, dolutegravir, fostemsavir, lamivudine, maraviroc, rilpiverine, tenofovir disoproxil, tenofovir, tenofovir afenamide, islatravir, doravirine, preziata, S-648414, GSK3640254, N6LS, GSK3739937/VH3739937, and S-365598.

In another embodiment, the invention provides a pharmaceutical composition comprising a compound of Formula Ia which is amorphous, and another therapeutic agent selected from the group consisting of abacavir, atazanavir, bictegravir, cabotegravir, dolutegravir, fostemsavir, lamivudine, maraviroc, rilpiverine, tenofovir disoproxil, tenofovir, tenofovir afenamide, islatravir, doravirine, preziata, S-648414, GSK3640254, N6LS, GSK3739937/VH3739937, and S-365598. In one embodiment, the invention provides a pharmaceutical composition comprising a compound of Formula Ib which is amorphous, and another therapeutic agent selected from the group consisting of abacavir, atazanavir, bictegravir, cabotegravir, dolutegravir, fostemsavir, lamivudine, maraviroc, rilpiverine, tenofovir disoproxil, tenofovir, tenofovir afenamide, islatravir, doravirine, preziata, S-648414, GSK3640254, N6LS, GSK3739937/VH3739937, and S-365598.

In one embodiment, the other agent is selected from the group consisting of, dolutegravir, lamivudine, fostemsavir, cabotegravir, N6LS, GSK3739937/VH3739937, GSK4000422/VH4000422, and S-365598.

In one embodiment, the invention provides a pharmaceutical composition comprising a compound of Formula Ia, or a pharmaceutically acceptable salt thereof, and another therapeutic agent selected from the group consisting of dolutegravir, lamivudine, fostemsavir, cabotegravir, N6LS, GSK3739937/VH3739937, GSK4000422/VH4000422, and S-365598. In one embodiment, the invention provides a pharmaceutical composition comprising a compound of Formula Ib, or a pharmaceutically acceptable salt thereof, and another therapeutic agent selected from the group consisting of dolutegravir, lamivudine, fostemsavir, cabotegravir, N6LS, GSK3739937/VH3739937, GSK4000422/VH4000422, and S-365598.

In one embodiment, the invention provides a pharmaceutical composition comprising a compound of Formula Ia and another therapeutic agent selected from the group consisting of dolutegravir, lamivudine, fostemsavir, cabotegravir, N6LS, GSK3739937/VH3739937, GSK4000422/VH4000422, and S-365598. In one embodiment, the invention provides a pharmaceutical composition comprising a compound of Formula Ib and another therapeutic agent selected from the group consisting of dolutegravir, lamivudine, fostemsavir, cabotegravir, N6LS, GSK3739937/VH3739937, GSK4000422/VH4000422, and S-365598.

In another embodiment, the invention provides a pharmaceutical composition comprising a compound of Formula Ia which is amorphous, and another therapeutic agent selected from the group consisting of dolutegravir, lamivudine, fostemsavir, cabotegravir, N6LS, GSK3739937/VH3739937, GSK4000422/VH4000422, and S-365598. In another embodiment, the invention provides a pharmaceutical composition comprising a compound of Formula Ib which is amorphous, and another therapeutic agent selected from the group consisting of dolutegravir, lamivudine, fostemsavir, cabotegravir, N6LS, GSK3739937/VH3739937, GSK4000422/VH4000422, and S-365598.

In another embodiment, the other agent is selected from the group consisting of dolutegravir, bictegravir, islatravir, lamivudine, fostemsavir, S-365598, and cabotegravir.

In one embodiment, the invention provides a pharmaceutical composition comprising a compound of Formula Ia, or a pharmaceutically acceptable salt thereof, and another therapeutic agent selected from the group consisting of dolutegravir, bictegravir, islatravir, lamivudine, fostemsavir, S-365598, and cabotegravir. In one embodiment, the invention provides a pharmaceutical composition comprising a compound of Formula Ib, or a pharmaceutically acceptable salt thereof, and another therapeutic agent selected from the group consisting of dolutegravir, bictegravir, islatravir, lamivudine, fostemsavir, S-365598, and cabotegravir.

In one embodiment, the invention provides a pharmaceutical composition comprising a compound of Formula Ia and another therapeutic agent selected from the group consisting of dolutegravir, bictegravir, islatravir, lamivudine, fostemsavir, S-365598, and cabotegravir. In one embodiment, the invention provides a pharmaceutical composition comprising a compound of Formula Ib and another therapeutic agent selected from the group consisting of dolutegravir, bictegravir, islatravir, lamivudine, fostemsavir, S-365598, and cabotegravir.

In another embodiment, the invention provides a pharmaceutical composition comprising a compound of Formula Ia which is amorphous, and another therapeutic agent selected from the group consisting of dolutegravir, bictegravir, islatravir, lamivudine, fostemsavir, S-365598, and cabotegravir. In another embodiment, the invention provides a pharmaceutical composition comprising a compound of Formula Ib which is amorphous, and another therapeutic agent selected from the group consisting of dolutegravir, bictegravir, islatravir, lamivudine, fostemsavir, S-365598, and cabotegravir.

In another embodiment, the other agent is selected from the group consisting of dolutegravir, bictegravir, S-365598, and cabotegravir.

In one embodiment, the invention provides a pharmaceutical composition comprising a compound of Formula Ia, or a pharmaceutically acceptable salt thereof, and another therapeutic agent selected from the group consisting of dolutegravir, bictegravir, S-365598, and cabotegravir. In one embodiment, the invention provides a pharmaceutical composition comprising a compound of Formula Ib, or a pharmaceutically acceptable salt thereof, and another therapeutic agent selected from the group consisting of dolutegravir, bictegravir, S-365598, and cabotegravir.

In one embodiment, the invention provides a pharmaceutical composition comprising a compound of Formula Ia and another therapeutic agent selected from the group consisting of dolutegravir, bictegravir, S-365598, and cabotegravir. In one embodiment, the invention provides a pharmaceutical composition comprising a compound of Formula Ib and another therapeutic agent selected from the group consisting of dolutegravir, bictegravir, S-365598, and cabotegravir.

In another embodiment, the invention provides a pharmaceutical composition comprising a compound of Formula Ia which is amorphous, and another therapeutic agent selected from the group consisting of dolutegravir, bictegravir, S-365598, and cabotegravir. In another embodiment, the invention provides a pharmaceutical composition comprising a compound of Formula Ib which is amorphous, and another therapeutic agent selected from the group consisting of dolutegravir, bictegravir, S-365598, and cabotegravir.

In another embodiment, the other agent is dolutegravir. In one embodiment, the invention provides a pharmaceutical composition comprising a compound of Formula Ia, or a pharmaceutically acceptable salt thereof, and another therapeutic agent is dolutegravir. In one embodiment, the invention provides a pharmaceutical composition comprising a compound of Formula Ib, or a pharmaceutically acceptable salt thereof, and another therapeutic agent is dolutegravir. In one embodiment, the invention provides a pharmaceutical composition comprising a compound of Formula Ia and another therapeutic agent is dolutegravir. In one embodiment, the invention provides a pharmaceutical composition comprising a compound of Formula Ib and another therapeutic agent is dolutegravir. In another embodiment, the invention provides a pharmaceutical composition comprising a compound of Formula Ia which is amorphous, and another therapeutic agent is dolutegravir. In another embodiment, the invention provides a pharmaceutical composition comprising a compound of Formula Ib which is amorphous, and another therapeutic agent is dolutegravir.

In another embodiment, the other agent is cabotegravir. In one embodiment, the invention provides a pharmaceutical composition comprising a compound of Formula Ia, or a pharmaceutically acceptable salt thereof, and another therapeutic agent is cabotegravir. In one embodiment, the invention provides a pharmaceutical composition comprising a compound of Formula Ib, or a pharmaceutically acceptable salt thereof, and another therapeutic agent is cabotegravir. In one embodiment, the invention provides a pharmaceutical composition comprising a compound of Formula Ia and another therapeutic agent is cabotegravir. In one embodiment, the invention provides a pharmaceutical composition comprising a compound of Formula Ib and another therapeutic agent is cabotegravir. In another embodiment, the invention provides a pharmaceutical composition comprising a compound of Formula Ia which is amorphous, and another therapeutic agent is cabotegravir. In another embodiment, the invention provides a pharmaceutical composition comprising a compound of Formula Ib which is amorphous, and another therapeutic agent is cabotegravir.

In still another embodiment, the other agent is S-365598. In one embodiment, the invention provides a pharmaceutical composition comprising a compound of Formula Ia, or a pharmaceutically acceptable salt thereof, and another therapeutic agent is S-365598. In one embodiment, the invention provides a pharmaceutical composition comprising a compound of Formula Ib, or a pharmaceutically acceptable salt thereof, and another therapeutic agent is S-365598. In one embodiment, the invention provides a pharmaceutical composition comprising a compound of Formula Ia and another therapeutic agent is S-365598. In one embodiment, the invention provides a pharmaceutical composition comprising a compound of Formula Ib and another therapeutic agent is S-365598. In another embodiment, the invention provides a pharmaceutical composition comprising a compound of Formula Ia which is amorphous, and another therapeutic agent is S-365598. In another embodiment, the invention provides a pharmaceutical composition comprising a compound of Formula Ib which is amorphous, and another therapeutic agent is S-365598.

It will be understood that GSK3640254 is a compound as described in Dicker I, Jeffrey J L, Protack T, et al.; GSK3640254 Is a Novel HIV-1 Maturation Inhibitor with an Optimized Virology Profile’ Antimicrob Agents Chemother. 2022; 66 (1): e0187621. doi: 10.1128/AAC.01876-21; GSK3739937, also known as VH3739937; is an HIV maturation inhibitor and the compound of clinical trial NCT04493684; N6LS: also known as VRC-HIVMAB091-00-AB, is a human monoclonal antibody and the compound of clinical trial NCT03538626; S-365598: is a third-generation HIV integrase strand-transfer inhibitor (INSTI) discovered by Shionogi; and S-648414 is the compound of clinical trial NCT04147715.

### EXAMPLES

### Formulation A (Heterogeneous Suspension) is Found in Table A.

TABLE A

| | | Quantity | | | | |
|---|---|---|---|---|---|---|
| Ingredient | Function | (mg/mL) | | Quantity (% w/v) | Quantity | |
| Cmpd of Formula Ia | Active agent | 300 | mg/mL | 30% | 600 | mg |
| Poloxamer 388 | Surfactant | 54 | mg/mL | 5.4% | 108 | mg |
| Mannitol | Tonicity adjuster | 35 | mg/mL | 3.5% | 70 | mg |
| Acetic Acid, Glacial | Buffer | 0.155 | mg/mL | 0.016% | 0.31 | mg |
| Sodium Acetate | Buffer | 0.625 | mg/mL | 0.063% | 1.25 | mg |

| | | | | |
|---|---|---|---|---|
| Water | Vehicle | QS ML | N/A | QS ML |
| Nitrogen | Processing aid | QS ML | N/A | QS ML |

### Preparation of Formulation A:

Sodium acetate (438.36 mg) and glacial acetic acid (104 uL) were dissolved in water (500 mL) to afford a 10 mM acetate buffer solution. The acetate buffer solution (440.41 g) was combined with Poloxamer 338 (34.88 g) and mannitol (24.43 g) and the resulting solution was filtered through a 0.2 μm filter. The pH of the solution was measured as pH 5.04. The solution (278.25 g) was combined with the compound of Formula Ia (110.25 g). The stirred suspension was maintained between 1-25° C. and was circulated at 45-145 ml/min through a wet bead mill (Netzsch Minicer) set at 5.5 m/s agitator tip speed, containing 0.3 mm YTZ grinding beads (Nikkato Corp) until the desired mean particle diameter of about 0.3 μm was achieved. The concentration of the formulation was about 300 mg/ml of a suspended amorphous form of the compound of Formula Ia, with 5.4 w/vol % P338 and 3.5 w/vol % mannitol and the remainder of the composition comprised of the aqueous acetate buffer described above.

### Formulation B (Heterogeneous Suspension) is Found in Table B.

TABLE B

| Ingredient | Function | Quantity (mg/mL) | | Quantity (% w/v) | Quantity | |
|---|---|---|---|---|---|---|
| Cmpd of Formula Ib | Active agent | 300 | mg/mL | 30% | 600 | mg |
| Poloxamer 388 | Surfactant | 54 | mg/mL | 5.4% | 108 | mg |
| Mannitol | Tonicity adjuster | 35 | mg/mL | 3.5% | 70 | mg |
| Acetic Acid, Glacial | Buffer | 0.41 | mg/mL | 0.041% | 0.31 | mg |
| Sodium Acetate | Buffer | 1.43 | mg/mL | 0.143% | 1.25 | mg |

| | | | | |
|---|---|---|---|---|
| Water | Vehicle | QS ML | N/A | QS mL |
| Nitrogen | Processing aid | QS ML | N/A | QS mL |

### Preparation of Formulation B:

Sodium acetate (435.72 mg) and glacial acetic acid (104 uL) were dissolved in water (500 mL) to afford a 10 mM acetate buffer solution. The acetate buffer solution (440.85 g) was combined with Poloxamer 338 (34.89 g) and mannitol (24.46 g) and the resulting solution was filtered through a 0.2 μm filter. The pH of the vehicle was measured as pH 5.02. The solution (278.25 g) was combined with the compound of Formula Ib (110.25 g). The stirred suspension was maintained between 1-25° C. and was circulated at 45-145 ml/min through a wet bead mill (Netzsch Minicer) set at 5.8 m/s agitator tip speed, containing 0.3 mm YTZ grinding beads (Nikkato Corp) until the desired mean particle diameter of about 0.2 μm was achieved. The concentration of the formulation was about 300 mg/ml of a suspended amorphous form of the compound of Formula Ib, with 5.4% w/vol P338 and 3.5% w/vol mannitol and the remainder of the composition comprised of the aqueous acetate buffer described above.

### Preparation of Formulation C:

A glass bottle equipped with a lid was charged with PEG200 (135 g) and the solution was heated to 45° C. with stirring. To the solution was slowly added the compound of Formula Ia (60 g) while stirring and heating were maintained. Following the addition, heating and stirring were maintained until a homogeneous solution was obtained. The solution was cooled to room temperature while stirring. To the bottle was added a solution of Lecithin (45 g, “Lipoid E80”, egg-based containing 80 weight % phosphatidylcholine) in ethanol (60 g, anhydrous). The mixture was stirred for 15 to 30 min. to afford a clear, homogeneous solution. The composition of the solution is 20 w/w % the compound of Formula Ia, 45 w/w % PEG200, 20 w/w % ethanol, and 15 w/w % lecithin.

### Preparation of Formulation D:

A glass bottle equipped with a lid was charged with PEG200 (67.5 g) and the solution was heated to 45° C. with stirring. To the solution was slowly added the compound of Formula Ib (45 g) while heating and stirring were maintained. Following the addition, heating and stirring were maintained until a homogeneous solution was obtained. The solution was cooled to room temperature while stirring. To the solution was added ethanol (37.5 g, anhydrous), and the mixture was stirred for 15 to 30 min. to afford a clear, homogeneous solution. The composition of the solution is 30 w/w % the compound of Formula Ib, 45 w/w % PEG200, and 25 w/w % ethanol.

### Preparation of Formulation E:

A glass bottle equipped with a lid was charged with PEG200 (150 g) and the solution was heated to 45° C. with stirring. To the solution was slowly added the compound of Formula Ia (90 g) while heating and stirring were maintained. Following the addition, heating and stirring were maintained until a homogeneous solution was obtained. The solution was cooled to room temperature while stirring. To the solution was added ethanol (60 g, anhydrous), and the mixture was stirred for 15 to 30 min. to afford a clear, homogeneous solution. The composition of the solution is 30 w/w % the compound of Formula Ia, 50 w/w % PEG200, and 20 w/w % ethanol; density=1.11 g/mL; viscosity=49.9 mPa-s.

### Preparation of Formulation F:

A glass bottle equipped with a lid was charged with PEG200 (3.99 mL), ethanol (0.52 mL) and water (0.67 mL), and the mixture was then vortexed. To the solution was slowly added the compound of Formula Ib (931 mg) and the mixture was then vortexed. The mixture was sonicated to afford a clear, homogeneous solution. The composition of the solution is PEG200 (69%), ethanol (6.3%), water (10.3%), compound of Formula Ib (14.3%).

### Preparation of Formulation G:

A glass bottle equipped with a lid was charged with PEG200 (2.54 mL), ethanol (0.52 mL), propylene glycol (0.73 mL), and water (0.52 mL), and the mixture was then vortexed. To the solution was slowly added the compound of Formula Ib (772 mg), and the mixture was then vortexed. The mixture was sonicated to afford a clear, homogeneous solution. The composition of the solution is PEG200 (53.7%), ethanol (7.7%), propylene glycol (14.2%), water (9.8%), compound of Formula Ib (14.5%).

### Preparation of Formulation H:

A glass bottle equipped with a lid was charged with PEG200 (1.75 mL), ethanol (0.35 mL), and sesame oil (1.40 mL), and the mixture was then vortexed. To the solution was slowly added the compound of Formula Ib (628 mg), and the mixture was then vortexed. The solution was sonicated to afford a clear, homogeneous solution. The composition of the solution is PEG200 (47.3%), ethanol (6.6%), sesame oil (31%), compound of Formula Ib (15.1%).

### Preparation of Formulation I:

Water (455.94 g) was combined with poloxamer 338 (31.26 g) and mannitol (25.07 g) and the resulting solution was filtered through a 0.2 μm filter to afford the “vehicle”. To the vehicle (247.63 g) was added the compound of Formula Ib (64.09 g). The stirred suspension maintained between 1-25° C. was circulated after the vehicle (50.40 g) at 50-145 mL/min through a wet bead mill (Netzsch Minicer) set at 5.8 m/s agitator tip speed containing 0.3 mm YTZ grinding beads (Nikkato Corp) until the desired mean particle diameter of about 0.78 μm was achieved. The concentration of the formulation was about 168.95 mg/ml of the compound of Formula Ib, with about 6.49% wt/vol P338 and about 5.2% wt/vol mannitol, with the remainder of the composition comprised of water.

### Preparation of Formulation J:

Water (455.13 g) was combined with poloxamer 338 (31.29 g) and mannitol (25.01 g) and the resulting solution was filtered through a 0.2 μm filter to afford the “vehicle”. To the vehicle (238.34 g) was added the compound of Formula Ia (64.51 g). The stirred suspension maintained between 1-25° C. was circulated after the vehicle (50.40 g) at 73-145 mL/min through a wet bead mill (Netzsch Minicer) set at 5.8 m/s agitator tip speed containing 0.3 mm YTZ grinding beads (Nikkato Corp) until the desired mean particle diameter of about 0.40 μm was achieved. The concentration of the formulation was about 177 mg/ml of the compound of Formula Ia, with about 6.3% wt/vol P338 and about 5.03% wt/vol mannitol, with the remainder of the composition comprised of water.

### Preparation of Formulation K:

To a mixing vessel, 1299.8 grams of PEG200 and 427.2 grams of ethanol were charged to a mixing vessel. The solution was stirred at ambient temperature for 15 minutes while gradually adding 40.85 grams of a compound of Formula Ia. The solution was stirred for approximately 2 hours until the compound was completely dissolved to yield a clear uniform solution. The resulting solution had a viscosity of 25 cP and a density of 1.089 g/mL.

### General Procedures for Analysis of Blood Samples:

### General Procedure A:

Blood samples were collected into K₂EDTA tubes, placed on water ice immediately after collection, and centrifuged as soon as possible to obtain plasma. Plasma samples were stored at −70° C. or colder until analysis by LC-MS/MS. All in vitro samples were injected on an MDS Sciex 5000 triple-quadrupole LC-MS/MS system. The analytical column used was a Waters Acquity 1.7 μm CSH Fluror Phenyl (2.1 mm×50 mm) maintained at 50° C. Mobile Phase A consisted of 0.1% (v/v) formic acid in MilliQ-purified water. Mobile Phase B consisted of 0.1% (v/v) formic acid in acetonitrile. The flow rate was 0.80 ml/min. The gradient was as follows: Mobile B was held for 0.2 minutes at 20% and then linearly increased from 20% to 75% over 0.4 min, followed by another linear increase from 75-95% over 0.55 min. It was then maintained at 95% for 0.35 min, and maintained at 20% for 0.49 min.

### General Procedure B:

Blood samples were collected into K₂EDTA tubes, placed on water ice immediately after collection, and centrifuged as soon as possible to obtain plasma. Plasma samples were stored at −70° C. or colder until analysis by LC-MS/MS. All in vitro samples were injected on a MDS Sciex 6500+ triple-quadrupole LC-MS/MS system. The analytical column used was a Waters Acquity 1.7 μm BEH (C18, 2.1 mm×50 mm, 1.7 μm) maintained at 35° C. Mobile Phase A consisted of 0.1% (v/v) formic acid in MilliQ-purified water. Mobile Phase B consisted of 0.1% (v/v) formic acid in acetonitrile. The flow rate was 0.80 mL/min. The gradient was as follows: Mobile B was held for 0.2 minutes at 2% and then linearly increased from 2% to 75% over 0.4 min, followed by another linear increase from 75-95% over 0.55 min. It was then maintained at 95% for 0.35 min, and maintained at 2% for 0.49 min.

### Procedure for Measuring Pharmacokinetic Parameters for Formulation A in an In Vivo Experiment

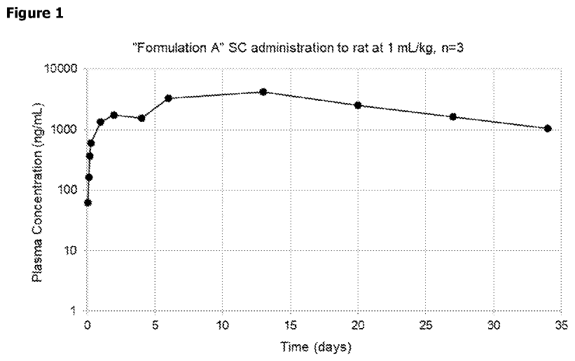

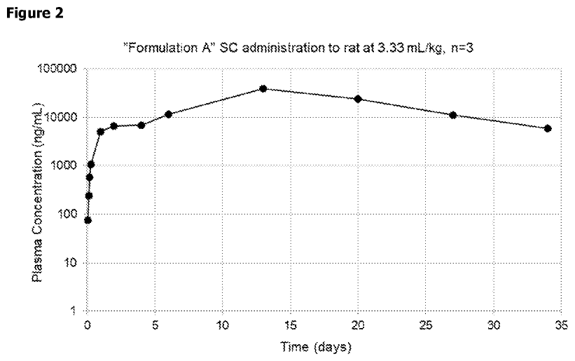

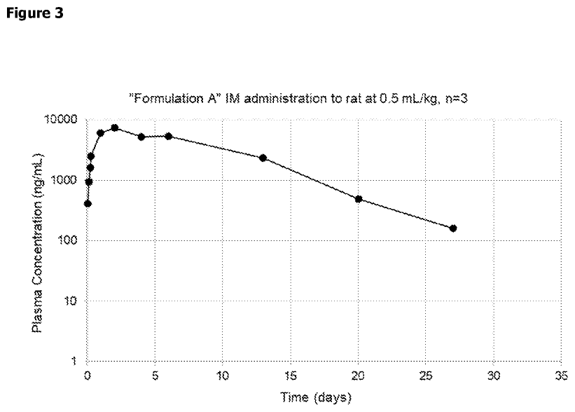

“Formulation A” was administered to Wistar Han Rats as either a subcutaneous injection at a dose of 1 ml/kg; a subcutaneous injection at a dose of 3.33 ml/kg; or as an intramuscular injection at a dose of 0.5 ml/kg. Blood samples were collected at the times indicated in Tables 1-3 and were analyzed according to General Procedure A. Results of the PK experiments are described in Tables 1-3 and FIGS. 1-3.

TABLE 1
Plasma concentration vs. time data for a study evaluating Formulation
A administered to rats subcutaneously at 1 mL/kg (n = 3).

| | | | | | Avg. | Std. Dev. |
|---|---|---|---|---|---|---|
| | | Rat 1 | Rat 2 | Rat 3 | Conc. | Conc. |
| Days | Hours | (ng/mL) | (ng/mL) | (ng/mL) | (ng/mL) | (ng/mL) |
| 0.04 | 1 | 52.7 | 83.6 | 48.5 | 62 | 19.2 |
| 0.13 | 3 | 166 | 172 | 152 | 163 | 10.3 |
| 0.21 | 5 | 309 | 439 | 356 | 368 | 65.8 |
| 0.29 | 7 | 576 | 577 | 620 | 591 | 25.1 |
| 1 | 24 | 1100 | 1350 | 1570 | 1340 | 235.2 |
| 2 | 48 | 1010 | 1810 | 2350 | 1723 | 674.2 |
| 4 | 96 | 671 | 1780 | 2110 | 1520 | 753.8 |
| 6 | 144 | 858 | 2490 | 6490 | 3279 | 2897.8 |
| 13 | 312 | 3050 | 5280 | 9130 | 4165 | 1576.8 |
| 20 | 480 | 2410 | 2580 | 2940 | 2495 | 120.2 |
| 27 | 648 | 1710 | 1480 | 1520 | 1595 | 162.6 |
| 34 | 816 | 1090 | 999 | 863 | 1045 | 64.3 |

LOD = Limit of detection

TABLE 2
Plasma concentration vs. time data for a study evaluating Formulation
A administered to rats subcutaneously at 3.33 mL/kg (n = 3).

| | | | | | Avg. | Std. Dev. |
|---|---|---|---|---|---|---|
| | | Rat 1 | Rat 2 | Rat 3 | Conc. | Conc. |
| Days | Hours | (ng/mL) | (ng/mL) | (ng/mL) | (ng/mL) | (ng/mL) |
| 0.04 | 1 | 107 | 67.5 | 52.7 | 76 | 28.1 |
| 0.13 | 3 | 284 | 275 | 173 | 244 | 61.7 |
| 0.21 | 5 | 627 | 730 | 396 | 584 | 171.0 |
| 0.29 | 7 | 983 | 1340 | 854 | 1059 | 251.8 |
| 1 | 24 | 5290 | 5940 | 3890 | 5040 | 1047.6 |
| 2 | 48 | 7900 | 7940 | 4460 | 6767 | 1997.7 |
| 4 | 96 | 6200 | 10800 | 3520 | 6840 | 3682.0 |
| 6 | 144 | 8300 | 19900 | 5950 | 11383 | 7468.7 |
| 13 | 312 | 31000 | 47400 | 37600 | 38667 | 8251.9 |
| 20 | 480 | 23300 | 27300 | 20600 | 23733 | 3371.0 |
| 27 | 648 | 11900 | 13200 | 8580 | 11227 | 2382.5 |
| 34 | 816 | 7120 | 6240 | 4350 | 5903 | 1415.4 |

LOD = Limit of detection

TABLE 3
Plasma concentration vs. time data for a study evaluating Formulation
A administered to rats intramuscularly at 0.5 mL/kg (n = 3).

| | | | | | Avg. | Std. Dev. |
|---|---|---|---|---|---|---|
| | | Rat 1 | Rat 2 | Rat 3 | Conc. | Conc. |
| Days | Hours | (ng/mL) | (ng/mL) | (ng/mL) | (ng/mL) | (ng/mL) |
| 0.04 | 1 | 247 | 356 | 640 | 414 | 202.9 |
| 0.13 | 3 | 598 | 1060 | 1160 | 939 | 299.8 |
| 0.21 | 5 | 1080 | 1920 | 1890 | 1630 | 476.6 |
| 0.29 | 7 | 1710 | 2880 | 2850 | 2480 | 667.0 |
| 1 | 24 | 5410 | 6840 | 5870 | 6040 | 730.0 |
| 2 | 48 | 6870 | 8480 | 6400 | 7250 | 1090.8 |
| 4 | 96 | 4910 | 6300 | 4520 | 5243 | 935.6 |
| 6 | 144 | 6260 | 5230 | 4590 | 5360 | 842.6 |
| 13 | 312 | 1650 | 1990 | 3390 | 2343 | 922.2 |
| 20 | 480 | 287 | 556 | 615 | 486 | 174.8 |
| 27 | 648 | 77.4 | 208 | 198 | 161 | 72.7 |

LOD = Limit of detection

### Procedure for Measuring Pharmacokinetic Parameters for Formulation B in an In Vivo Experiment

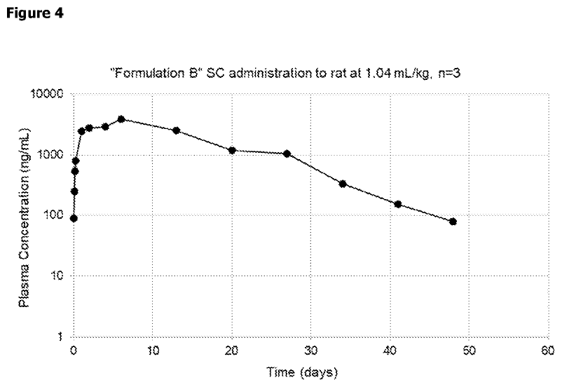

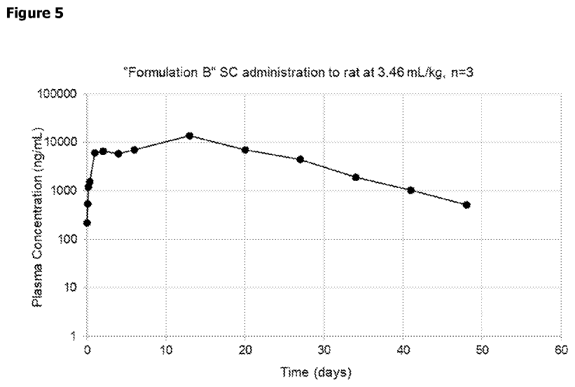

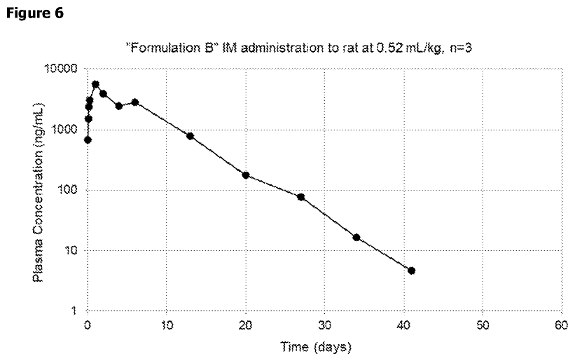

“Formulation B” was administered to Wistar Han Rats as a either a subcutaneous injection at a dose of 1.04 mL/kg; a subcutaneous injection at a dose of 3.46 ml/kg; or as an intramuscular injection at a dose of 0.52 mL/kg. Blood samples were collected at the times indicated in Tables 4-6 and were analyzed according to General Procedure B. Results of the PK experiments are described in Tables 4-6 and FIGS. 4-6.

TABLE 4
Plasma concentration vs. time data for a study evaluating Formulation
B administered to rats subcutaneously at 1.04 mL/kg (n = 3).

| | | | | | Avg. | Std. Dev. |
|---|---|---|---|---|---|---|
| | | Rat 1 | Rat 2 | Rat 3 | Conc. | Conc. |
| Days | Hours | (ng/mL) | (ng/mL) | (ng/mL) | (ng/mL) | (ng/mL) |
| 0.04 | 1 | 113.0 | 82.7 | 75.2 | 90.3 | 20.0 |
| 0.13 | 3 | 326.0 | 214.0 | 208.0 | 249.3 | 66.5 |
| 0.21 | 5 | 600.0 | 558.0 | 461.0 | 539.7 | 71.3 |
| 0.29 | 7 | 894.0 | 733.0 | 754.0 | 793.7 | 87.5 |
| 1 | 24 | 2200.0 | 2500.0 | 2640.0 | 2446.7 | 224.8 |
| 2 | 48 | 3120.0 | 2790.0 | 2310.0 | 2740.0 | 407.3 |
| 4 | 96 | 3780.0 | 3020.0 | 1900.0 | 2900.0 | 945.7 |
| 6 | 144 | 5190.0 | 3400.0 | 2970.0 | 3853.3 | 1177.4 |
| 13 | 312 | 2120.0 | 2150.0 | 3340.0 | 2536.7 | 695.9 |
| 20 | 480 | 808.0 | 1110.0 | 1590.0 | 1169.3 | 394.4 |
| 27 | 648 | 685.0 | 985.0 | 1500.0 | 1056.7 | 412.2 |
| 34 | 816 | 175.0 | 369.0 | 462.0 | 335.3 | 146.4 |
| 41 | 984 | 92.2 | 125.0 | 242.0 | 153.1 | 78.7 |
| 48 | 1152 | 47.5 | 55.0 | 133.0 | 78.5 | 47.3 |

LOD = Limit of detection

TABLE 5
Plasma concentration vs. time data for a study evaluating Formulation
B administered to rats subcutaneously at 3.46 mL/kg (n = 3).

| | | | | | Avg. | Std. Dev. |
|---|---|---|---|---|---|---|
| | | Rat 1 | Rat 2 | Rat 3 | Conc. | Conc. |
| Days | Hours | (ng/mL) | (ng/mL) | (ng/mL) | (ng/mL) | (ng/mL) |
| 0.04 | 1 | 220.0 | 260.0 | 168.0 | 216.0 | 46.1 |
| 0.13 | 3 | 580.0 | 587.0 | 454.0 | 540.3 | 74.8 |
| 0.21 | 5 | 1120.0 | 1420.0 | 1120.0 | 1220.0 | 173.2 |
| 0.29 | 7 | 1360.0 | 1750.0 | 1440.0 | 1516.7 | 206.0 |
| 1 | 24 | 6480.0 | 7390.0 | 4200.0 | 6023.3 | 1643.3 |
| 2 | 48 | 10300.0 | 5720.0 | 3610.0 | 6543.3 | 3420.2 |
| 4 | 96 | 10800.0 | 4280.0 | 2680.0 | 5920.0 | 4301.3 |
| 6 | 144 | 9620.0 | 6520.0 | 4710.0 | 6950.0 | 2483.1 |
| 13 | 312 | 16100.0 | 13700.0 | 11200.0 | 13666.7 | 2450.2 |
| 20 | 480 | 4990.0 | 8720.0 | 7310.0 | 7006.7 | 1883.4 |
| 27 | 648 | 3090.0 | 4580.0 | 5530.0 | 4400.0 | 1229.9 |
| 34 | 816 | 1200.0 | 1720.0 | 2860.0 | 1926.7 | 849.1 |
| 41 | 984 | 442.0 | 877.0 | 1820.0 | 1046.3 | 704.4 |
| 48 | 1152 | 141.0 | 317.0 | 1080.0 | 512.7 | 499.1 |

LOD = Limit of detection

TABLE 6
Plasma concentration vs. time data for a study evaluating Formulation
B administered to rats intramuscularly at 0.52 mL/kg (n = 3).

| | | | | | Avg. | Std. Dev. |
|---|---|---|---|---|---|---|
| | | Rat 1 | Rat 2 | Rat 3 | Conc. | Conc. |
| Days | Hours | (ng/mL) | (ng/mL) | (ng/mL) | (ng/mL) | (ng/mL) |
| 0.04 | 1 | 386.0 | 942.0 | 680.0 | 669.3 | 278.2 |
| 0.13 | 3 | 1020.0 | 1790.0 | 1690.0 | 1500.0 | 418.7 |
| 0.21 | 5 | 1200.0 | 3370.0 | 2490.0 | 2353.3 | 1091.4 |
| 0.29 | 7 | 1630.0 | 4990.0 | 2580.0 | 3066.7 | 1732.1 |
| 1 | 24 | 4050.0 | 7750.0 | 4950.0 | 5583.3 | 1929.6 |
| 2 | 48 | 2380.0 | 5720.0 | 3560.0 | 3886.7 | 1693.8 |
| 4 | 96 | 2690.0 | 2340.0 | 2320.0 | 2450.0 | 208.1 |
| 6 | 144 | 3000.0 | 2090.0 | 3300.0 | 2796.7 | 630.1 |
| 13 | 312 | 1150.0 | 761.0 | 448.0 | 786.3 | 351.7 |
| 20 | 480 | 219.0 | 189.0 | 119.0 | 175.7 | 51.3 |
| 27 | 648 | 94.3 | 64.8 | 69.8 | 76.3 | 15.8 |
| 34 | 816 | 24.2 | 16.2 | 8.9 | 16.4 | 7.7 |
| 41 | 984 | 4.7 | 4.6 | <LOD | 4.6 | N/A |
| 48 | 1152 | <LOD | <LOD | <LOD | <LOD | N/A |

LOD = Limit of detection

### Procedure for Measuring Pharmacokinetic Parameters for Formulation C in an In Vivo Experiment

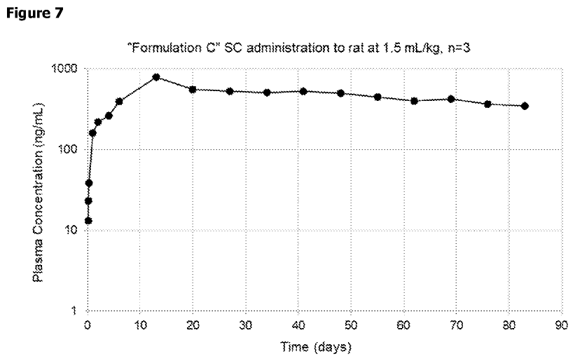

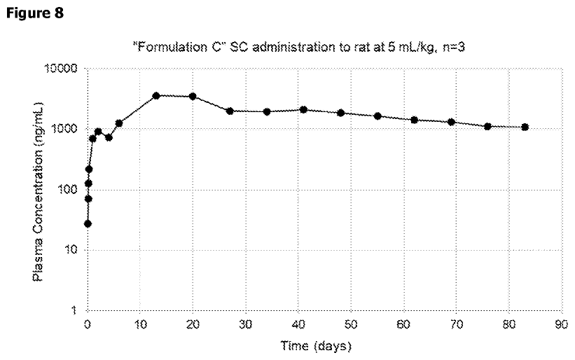

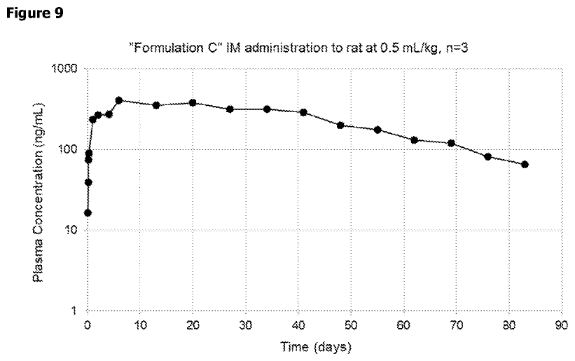

“Formulation C” was administered to Wistar Han Rats as either a subcutaneous injection at a dose of 1.5 mL/kg; a subcutaneous injection at a dose of 5 ml/kg; or as an intramuscular injection at a dose of 0.5 mL/kg. Blood samples were collected at the times indicated in Tables 7-9 and were analyzed according to General Procedure A. Results of the PK experiments are described in Tables 7-9 and FIGS. 7-9.

TABLE 7
Plasma concentration vs. time data for a study evaluating Formulation
C administered to rats subcutaneously at 1.5 mL/kg (n = 3).

| | | | | | Avg. | Std. Dev. |
|---|---|---|---|---|---|---|
| | | Rat 1 | Rat 2 | Rat 3 | Conc. | Conc. |
| Days | Hours | (ng/mL) | (ng/mL) | (ng/mL) | (ng/mL) | (ng/mL) |
| 0.04 | 1 | | | | | |
| 0.13 | 3 | 15.5 | 10.8 | | 13.15 | 3.3 |
| 0.21 | 5 | 18.7 | 25.2 | 26 | 23.3 | 4.0 |
| 0.29 | 7 | 25.9 | 48.4 | 42.4 | 38.9 | 11.7 |
| 1 | 24 | 145 | 148 | 187 | 160 | 23.4 |
| 2 | 48 | 209 | 209 | 241 | 220 | 18.5 |
| 4 | 96 | 294 | 298 | 198 | 263 | 56.6 |
| 6 | 144 | 400 | 536 | 245 | 394 | 145.6 |
| 13 | 312 | 746 | 748 | 859 | 784 | 64.7 |
| 20 | 480 | 749 | 460 | 460 | 556 | 166.9 |
| 27 | 648 | 656 | 473 | 460 | 530 | 109.6 |
| 34 | 816 | 603 | 440 | 484 | 509 | 84.3 |
| 41 | 984 | 617 | 510 | 459 | 529 | 80.6 |
| 48 | 1152 | 545 | 472 | 477 | 498 | 40.8 |
| 55 | 1320 | 569 | 362 | 420 | 450 | 106.8 |
| 62 | 1488 | 423 | 369 | 421 | 404 | 30.6 |
| 69 | 1656 | 477 | 384 | 407 | 423 | 48.4 |
| 76 | 1824 | 392 | 321 | 385 | 366 | 39.1 |
| 83 | 1992 | 375 | 327 | 344 | 349 | 24.3 |

LOD = Limit of detection

TABLE 8
Plasma concentration vs. time data for a study evaluating Formulation
C administered to rats subcutaneously at 5 mL/kg (n = 3).

| | | | | | | Std. Dev. |
|---|---|---|---|---|---|---|
| | | Rat 1 | Rat 2 | Rat 3 | Avg. Conc. | Conc. |
| Days | Hours | (ng/mL) | (ng/mL) | (ng/mL) | (ng/mL) | (ng/mL) |
| 0.04 | 1 | | 37.3 | 17.5 | 27 | 14.0 |
| 0.13 | 3 | 15.6 | 135 | 59.3 | 70 | 60.4 |
| 0.21 | 5 | 36.6 | 202 | 142 | 127 | 83.7 |
| 0.29 | 7 | 75.8 | 328 | 245 | 216 | 128.5 |
| 1 | 24 | 314 | 1020 | 765 | 700 | 357.5 |
| 2 | 48 | 446 | 1040 | 1240 | 909 | 413.0 |
| 4 | 96 | 404 | 681 | 1120 | 735 | 361.0 |
| 6 | 144 | 932 | 1060 | 1800 | 1264 | 468.6 |
| 13 | 312 | 3910 | 3220 | 3490 | 3540 | 347.7 |
| 20 | 480 | 3720 | 3790 | 3040 | 3517 | 414.3 |
| 27 | 648 | 2750 | 2000 | 1250 | 2000 | 750.0 |
| 34 | 816 | 2530 | 2140 | 1160 | 1943 | 705.9 |
| 41 | 984 | 2980 | 2260 | 974 | 2071 | 1016.2 |
| 48 | 1152 | 2710 | 2010 | 841 | 1854 | 944.3 |
| 55 | 1320 | 2320 | 1840 | 769 | 1643 | 794.0 |
| 62 | 1488 | 1810 | 1710 | 671 | 1397 | 630.7 |
| 69 | 1656 | 1780 | 1490 | 695 | 1322 | 561.7 |
| 76 | 1824 | 1410 | 1390 | 555 | 1118 | 488.0 |
| 83 | 1992 | 1350 | 1360 | 517 | 1076 | 483.8 |

LOD = Limit of detection

TABLE 9
Plasma concentration vs. time data for a study evaluating Formulation
C administered to rats intramuscularly at 0.5 mL/kg (n = 3).

| | | | | | | Std. Dev. |
|---|---|---|---|---|---|---|
| | | Rat 1 | Rat 2 | Rat 3 | Avg. Conc. | Conc. |
| Days | Hours | (ng/mL) | (ng/mL) | (ng/mL) | (ng/mL) | (ng/mL) |
| 0.04 | 1 | 21.6 | 11 | | 16 | 7.5 |
| 0.13 | 3 | 73.8 | 30.8 | 13.2 | 39 | 31.2 |
| 0.21 | 5 | 125 | 71.9 | 27.6 | 75 | 48.8 |
| 0.29 | 7 | 128 | 95.7 | 46.6 | 90 | 41.0 |
| 1 | 24 | 362 | 201 | 150 | 238 | 110.7 |
| 2 | 48 | 334 | 317 | 153 | 268 | 100.0 |
| 4 | 96 | 400 | 276 | 145 | 274 | 127.5 |
| 6 | 144 | 556 | 442 | 236 | 411 | 162.2 |
| 13 | 312 | 495 | 376 | 196 | 356 | 150.5 |
| 20 | 480 | 531 | 411 | 205 | 382 | 164.9 |
| 27 | 648 | 376 | 438 | 140 | 318 | 157.2 |
| 34 | 816 | 407 | 384 | 155 | 315 | 139.3 |
| 41 | 984 | 325 | 405 | 133 | 288 | 139.8 |
| 48 | 1152 | 226 | 279 | 100 | 202 | 91.9 |
| 55 | 1320 | 186 | 249 | 91.3 | 175 | 79.4 |
| 62 | 1488 | 128 | 204 | 62.6 | 132 | 70.8 |
| 69 | 1656 | 119 | 182 | 62.2 | 121 | 59.9 |
| 76 | 1824 | 85.2 | 115 | 45 | 82 | 35.1 |
| 83 | 1992 | 66.1 | 102 | 31 | 66 | 35.5 |

LOD = Limit of detection

### Procedure for Measuring Pharmacokinetic Parameters for Formulation D in an In Vivo Experiment

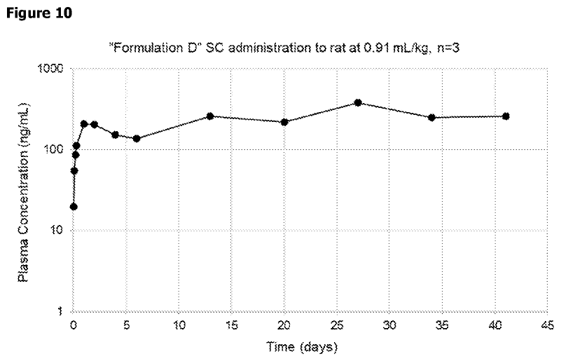

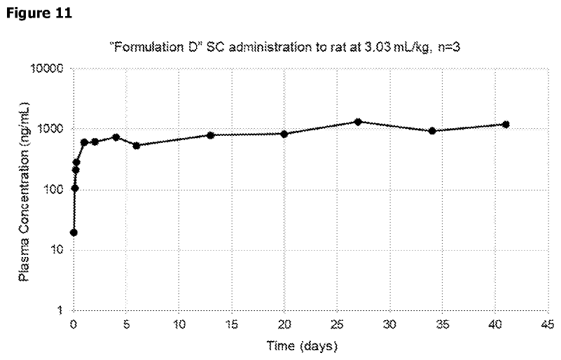

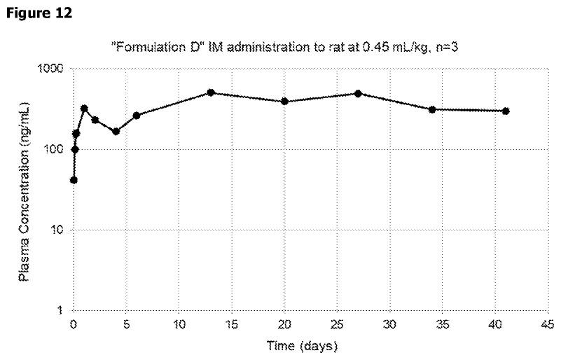

“Formulation D” was administered to Wistar Han Rats as either a subcutaneous injection at a dose of 0.91 ml/kg; a subcutaneous injection at a dose of 3.03 ml/kg; or as an intramuscular injection at a dose of 0.45 ml/kg. Blood samples were collected at the times indicated in Tables 10-12 and were analyzed according to General Procedure B. Results of the PK experiments are described in Tables 10-12 and FIGS. 10-12.

TABLE 10
Plasma concentration vs. time data for a study evaluating Formulation
D administered to rats subcutaneously at 0.91 mL/kg (n = 3).

| | | | | | | Std. Dev. |
|---|---|---|---|---|---|---|
| | | Rat 1 | Rat 2 | Rat 3 | Avg. Conc. | Conc. |
| Days | Hours | (ng/mL) | (ng/mL) | (ng/mL) | (ng/mL) | (ng/mL) |
| 0.04 | 1 | 46.9 | 4.8 | 7.2 | 19.6 | 23.6 |
| 0.13 | 3 | 118.0 | 22.7 | 24.4 | 55.0 | 54.5 |
| 0.21 | 5 | 171.0 | 46.4 | 41.4 | 86.3 | 73.4 |
| 0.29 | 7 | 212.0 | 62.9 | 62.2 | 112.4 | 86.3 |
| 1 | 24 | 330.0 | 146.0 | 147.0 | 207.7 | 105.9 |
| 2 | 48 | 113.0 | 153.0 | 354.0 | 206.7 | 129.2 |
| 4 | 96 | 253.0 | 92.0 | 114.0 | 153.0 | 87.3 |
| 6 | 144 | 268.0 | 53.9 | 90.3 | 137.4 | 114.6 |
| 13 | 312 | 200.0 | 180.0 | 406.0 | 262.0 | 125.1 |
| 20 | 480 | 282.0 | 185.0 | 195.0 | 220.7 | 53.4 |
| 27 | 648 | 507.0 | 344.0 | 288.0 | 379.7 | 113.8 |
| 34 | 816 | 289.0 | 248.0 | 211.0 | 249.3 | 39.0 |
| 41 | 984 | 302.0 | 223.0 | 254.0 | 259.7 | 39.8 |

LOD = Limit of detection

TABLE 11
Plasma concentration vs. time data for a study evaluating Formulation
D administered to rats subcutaneously at 3.03 mL/kg (n = 3).

| | | | | | | Std. Dev. |
|---|---|---|---|---|---|---|
| | | Rat 1 | Rat 2 | Rat 3 | Avg. Conc. | Conc. |
| Days | Hours | (ng/mL) | (ng/mL) | (ng/mL) | (ng/mL) | (ng/mL) |
| 0.04 | 1 | 10.3 | 32.4 | 15.7 | 19.5 | 11.5 |
| 0.13 | 3 | 70.5 | 169.0 | 79.7 | 106.4 | 54.4 |
| 0.21 | 5 | 176.0 | 296.0 | 167.0 | 213.0 | 72.0 |
| 0.29 | 7 | 222.0 | 369.0 | 263.0 | 284.7 | 75.9 |
| 1 | 24 | 470.0 | 816.0 | 514.0 | 600.0 | 188.4 |
| 2 | 48 | 535.0 | 785.0 | 511.0 | 610.3 | 151.7 |
| 4 | 96 | 506.0 | 1140.0 | 557.0 | 734.3 | 352.2 |
| 6 | 144 | 301.0 | 837.0 | 462.0 | 533.3 | 275.0 |
| 13 | 312 | 701.0 | 1000.0 | 664.0 | 788.3 | 184.2 |
| 20 | 480 | 637.0 | 1200.0 | 663.0 | 833.3 | 317.8 |
| 27 | 648 | 1210.0 | 1930.0 | 822.0 | 1320.7 | 562.2 |
| 34 | 816 | 776.0 | 1280.0 | 717.0 | 924.3 | 309.4 |
| 41 | 984 | 1210.0 | 1540.0 | 828.0 | 1192.7 | 356.3 |

LOD = Limit of detection

TABLE 12
Plasma concentration vs. time data for a study evaluating Formulation
D administered to rats intramuscularly at 0.45 mL/kg (n = 3).

| | | | | | | Std. Dev. |
|---|---|---|---|---|---|---|
| | | Rat 1 | Rat 2 | Rat 3 | Avg. Conc. | Conc. |
| Days | Hours | (ng/mL) | (ng/mL) | (ng/mL) | (ng/mL) | (ng/mL) |
| 0.04 | 1 | 55.8 | 38.9 | 28.5 | 41.1 | 13.8 |
| 0.13 | 3 | 167.0 | 79.0 | 53.9 | 100.0 | 59.4 |
| 0.21 | 5 | 261.0 | 127.0 | 72.0 | 153.3 | 97.2 |
| 0.29 | 7 | 248.0 | 136.0 | 92.8 | 158.9 | 80.1 |
| 1 | 24 | 516.0 | 287.0 | 156.0 | 319.7 | 182.2 |
| 2 | 48 | 331.0 | 247.0 | 118.0 | 232.0 | 107.3 |
| 4 | 96 | 233.0 | 182.0 | 81.8 | 165.6 | 76.9 |
| 6 | 144 | 358.0 | 288.0 | 146.0 | 264.0 | 108.0 |
| 13 | 312 | 495.0 | 785.0 | 228.0 | 502.7 | 278.6 |
| 20 | 480 | 381.0 | 594.0 | 194.0 | 389.7 | 200.1 |
| 27 | 648 | 513.0 | 685.0 | 273.0 | 490.3 | 206.9 |
| 34 | 816 | 321.0 | 426.0 | 179.0 | 308.7 | 124.0 |
| 41 | 984 | 293.0 | 426.0 | 173.0 | 297.3 | 126.6 |

LOD = Limit of detection

### Procedure for Measuring Pharmacokinetic Parameters for Formulation F in an In Vivo Experiment

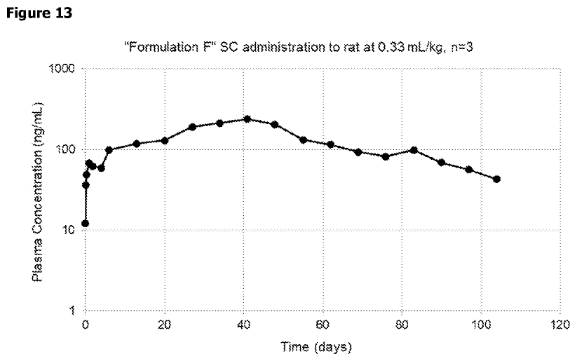

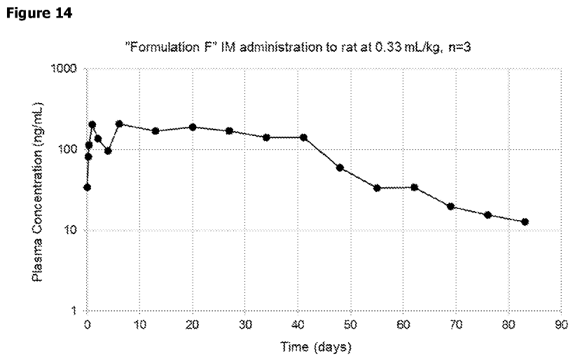

“Formulation F” was administered to Wistar Han Rats as either a subcutaneous injection at a dose of 0.33 mL/kg or as an intramuscular injection at a dose of 0.33 mL/kg. Blood samples were collected at the times indicated in Tables 13-14 and were analyzed according to General Procedure B. Results of the PK experiments are described in Tables 13-14 and FIGS. 13-14.

TABLE 13
Plasma concentration vs. time data for a study evaluating Formulation
F administered to rats subcutaneously at 0.33 mL/kg (n = 3).

| | | | | | | Std. Dev. |
|---|---|---|---|---|---|---|
| | | Rat 1 | Rat 2 | Rat 3 | Avg. Conc. | Conc. |
| Days | Hours | (ng/mL) | (ng/mL) | (ng/mL) | (ng/mL) | (ng/mL) |
| 0.0 | 1.0 | 16.1 | 12.3 | 8.2 | 12.2 | 4.0 |
| 0.2 | 4.0 | 39.7 | 46.2 | 22.8 | 36.2 | 12.1 |
| 0.3 | 7.0 | 53.2 | 68.2 | 25.3 | 48.9 | 21.8 |
| 1 | 24 | 75.4 | 81.0 | 49.1 | 68.5 | 17.0 |
| 2 | 48 | 59.1 | 83.2 | 45.0 | 62.4 | 19.3 |
| 4 | 96 | 59.2 | 64.8 | 52.6 | 58.9 | 6.1 |
| 6 | 144 | 77.5 | 166.0 | 51.7 | 98.4 | 59.9 |
| 13 | 312 | 88.4 | 159.0 | 105.0 | 117.5 | 36.9 |
| 20 | 480 | 93.5 | 186.0 | 108.0 | 129.2 | 49.8 |
| 27 | 648 | 173.0 | 241.0 | 157.0 | 190.3 | 44.6 |
| 34 | 816 | 210.0 | 245.0 | 180.0 | 211.7 | 32.5 |
| 41 | 984 | 179.0 | 266.0 | 270.0 | 238.3 | 51.4 |
| 48 | 1152 | 172.0 | 195.0 | 245.0 | 204.0 | 37.3 |
| 55 | 1320 | 107.0 | 122.0 | 165.0 | 131.3 | 30.1 |
| 62 | 1488 | 87.0 | 106.0 | 151.0 | 114.7 | 32.9 |
| 69 | 1656 | 78.6 | 78.2 | 123.0 | 93.3 | 25.8 |
| 76 | 1824 | 62.6 | 66.9 | 117.0 | 82.2 | 30.2 |
| 83 | 1992 | 90.4 | 80.4 | 125.0 | 98.6 | 23.4 |
| 90 | 2160 | 64.4 | 57.1 | 84.7 | 68.7 | 14.3 |
| 97 | 2328 | 45.0 | 49.3 | 75.1 | 56.5 | 16.3 |
| 104 | 2496 | 35.7 | 37.5 | 54.7 | 42.6 | 10.5 |

LOD = Limit of detection

TABLE 14
Plasma concentration vs. time data for a study evaluating Formulation
F administered to rats intramuscularly at 0.33 mL/kg (n = 3).

| | | | | | | Std. Dev. |
|---|---|---|---|---|---|---|
| | | Rat 1 | Rat 2 | Rat 3 | Avg. Conc. | Conc. |
| Days | Hours | (ng/mL) | (ng/mL) | (ng/mL) | (ng/mL) | (ng/mL) |
| 0.0 | 1.0 | 30.5 | 25.5 | 45.5 | 33.8 | 10.4 |
| 0.2 | 4.0 | 69.7 | 63.4 | 110.0 | 81.0 | 25.3 |
| 0.3 | 7.0 | 91.3 | 99.2 | 148.0 | 112.8 | 30.7 |
| 1 | 24 | 175.0 | 153.0 | 280.0 | 202.7 | 67.9 |
| 2 | 48 | 119.0 | 120.0 | 168.0 | 135.7 | 28.0 |
| 4 | 96 | 96.0 | 71.7 | 122.0 | 96.6 | 25.2 |
| 6 | 144 | 297.0 | 99.2 | 228.0 | 208.1 | 100.4 |
| 13 | 312 | 240.0 | 76.6 | 189.0 | 168.5 | 83.6 |
| 20 | 480 | 254.0 | 105.0 | 211.0 | 190.0 | 76.7 |
| 27 | 648 | 248.0 | 89.3 | 170.0 | 169.1 | 79.4 |
| 34 | 816 | 135.0 | 82.5 | 209.0 | 142.2 | 63.6 |
| 41 | 984 | 111.0 | 58.5 | 256.0 | 141.8 | 102.3 |
| 48 | 1152 | 63.6 | 34.7 | 80.4 | 59.6 | 23.1 |
| 55 | 1320 | 28.1 | 18.1 | 54.8 | 33.7 | 19.0 |
| 62 | 1488 | 25.4 | 13.4 | 62.6 | 33.8 | 25.7 |
| 69 | 1656 | 18.5 | 9.3 | 31.5 | 19.8 | 11.2 |
| 76 | 1824 | 14.9 | 6.7 | 25.0 | 15.5 | 9.2 |
| 83 | 1992 | 12.4 | 4.5 | 21.0 | 12.6 | 8.2 |

LOD = Limit of detection

### Procedure for Measuring Pharmacokinetic Parameters for Formulation G in an In Vivo Experiment

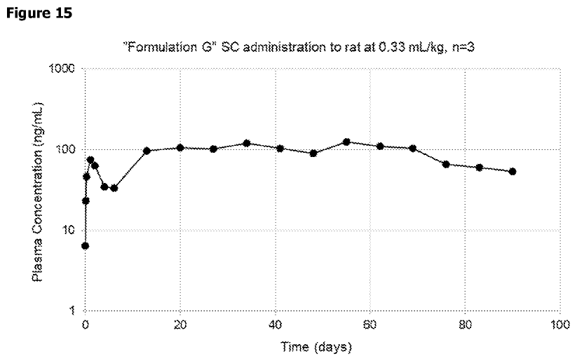

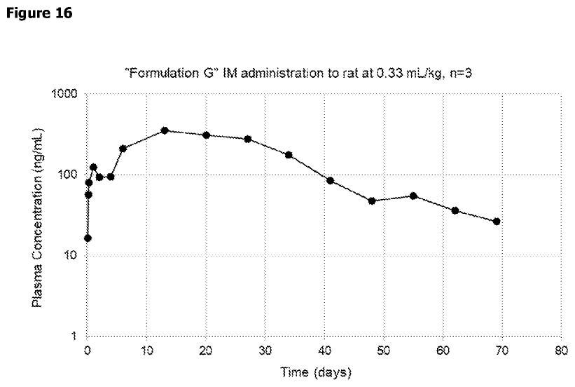

“Formulation G” was administered to Wistar Han Rats as either a subcutaneous injection at a dose of 0.33 mL/kg or as an intramuscular injection at a dose of 0.33 ml/kg. Blood samples were collected at the times indicated in Tables 15-16 and were analyzed according to General Procedure B. Results of the PK experiments are described in Tables 15-16 and FIGS. 15-16.

TABLE 15
Plasma concentration vs. time data for a study evaluating Formulation
G administered to rats subcutaneously at 0.33 mL/kg (n = 3).

| | | | | | | Std. Dev. |
|---|---|---|---|---|---|---|
| | | Rat 1 | Rat 2 | Rat 3 | Avg. Conc. | Conc. |
| Days | Hours | (ng/mL) | (ng/mL) | (ng/mL) | (ng/mL) | (ng/mL) |
| 0.0 | 1.0 | 5.1 | 8.3 | 6.0 | 6.5 | 1.7 |
| 0.2 | 4.0 | 19.1 | 29.1 | 21.9 | 23.4 | 5.2 |
| 0.3 | 7.0 | 39.0 | 56.6 | 43.0 | 46.2 | 9.2 |
| 1 | 24 | 57.0 | 103.0 | 63.2 | 74.4 | 25.0 |
| 2 | 48 | 48.1 | 78.9 | 62.2 | 63.1 | 15.4 |
| 4 | 96 | 25.1 | 40.1 | 39.3 | 34.8 | 8.4 |
| 6 | 144 | 21.1 | 29.6 | 49.1 | 33.3 | 14.4 |
| 13 | 312 | 102.0 | 72.7 | 116.0 | 96.9 | 22.1 |
| 20 | 480 | 136.0 | 52.9 | 125.0 | 104.6 | 45.1 |
| 27 | 648 | 87.6 | 72.7 | 146.0 | 102.1 | 38.7 |
| 34 | 816 | 108.0 | 65.6 | 183.0 | 118.9 | 59.4 |
| 41 | 984 | 109.0 | 54.0 | 146.0 | 103.0 | 46.3 |
| 48 | 1152 | 98.4 | 61.2 | 110.0 | 89.9 | 25.5 |
| 55 | 1320 | 141.0 | 92.7 | 140.0 | 124.6 | 27.6 |
| 62 | 1488 | 121.0 | 86.8 | 119.0 | 108.9 | 19.2 |
| 69 | 1656 | 107.0 | 101.0 | 103.0 | 103.7 | 3.1 |
| 76 | 1824 | 82.4 | 65.3 | 50.2 | 66.0 | 16.1 |
| 83 | 1992 | 80.6 | 57.1 | 43.5 | 60.4 | 18.8 |
| 90 | 2160 | 71.8 | 51.2 | 38.9 | 54.0 | 16.6 |

LOD = Limit of detection

TABLE 16
Plasma concentration vs. time data for a study evaluating Formulation
G administered to rats intramuscularly at 0.33 mL/kg (n = 3).

| | | | | | | Std. Dev. |
|---|---|---|---|---|---|---|
| | | Rat 1 | Rat 2 | Rat 3 | Avg. Conc. | Conc. |
| Days | Hours | (ng/mL) | (ng/mL) | (ng/mL) | (ng/mL) | (ng/mL) |
| 0.0 | 1.0 | 24.9 | 10.5 | 14.2 | 16.5 | 7.5 |
| 0.2 | 4.0 | 61.2 | 38.8 | 71.6 | 57.2 | 16.8 |
| 0.3 | 7.0 | 110.0 | 56.6 | 77.8 | 81.5 | 26.9 |
| 1 | 24 | 155.0 | 119.0 | 103.0 | 125.7 | 26.6 |
| 2 | 48 | 108.0 | 74.4 | 98.2 | 93.5 | 17.3 |
| 4 | 96 | 123.0 | 75.5 | 85.8 | 94.8 | 25.0 |
| 6 | 144 | 269.0 | 161.0 | 203.0 | 211.0 | 54.4 |
| 13 | 312 | 402.0 | 292.0 | 366.0 | 353.3 | 56.1 |
| 20 | 480 | 343.0 | 338.0 | 261.0 | 314.0 | 46.0 |
| 27 | 648 | 224.0 | 276.0 | 334.0 | 278.0 | 55.0 |
| 34 | 816 | 147.0 | 215.0 | 169.0 | 177.0 | 34.7 |
| 41 | 984 | 71.3 | 99.6 | 84.9 | 85.3 | 14.2 |
| 48 | 1152 | 30.2 | 68.3 | 44.9 | 47.8 | 19.2 |
| 55 | 1320 | 54.9 | 74.4 | 37.1 | 55.5 | 18.7 |
| 62 | 1488 | 30.8 | 50.9 | 25.8 | 35.8 | 13.3 |
| 69 | 1656 | 22.8 | 38.1 | 18.1 | 26.3 | 10.5 |

LOD = Limit of detection

### Procedure for Measuring Pharmacokinetic Parameters for Formulation H in an In Vivo Experiment

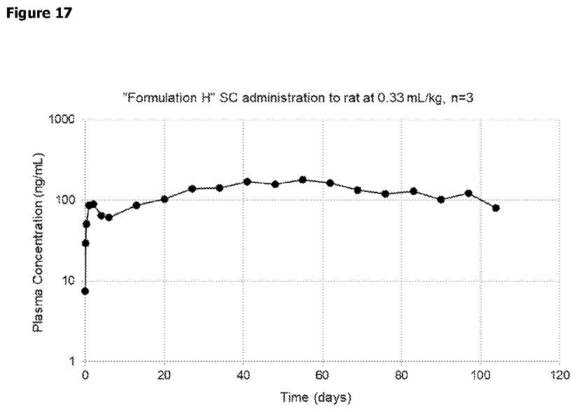

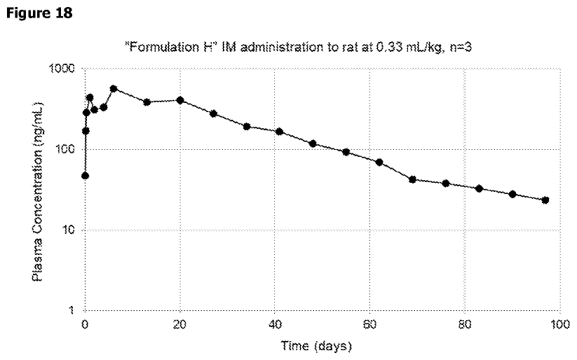

“Formulation H” was administered to Wistar Han Rats as either a subcutaneous injection at a dose of 0.33 mL/kg or as an intramuscular injection at a dose of 0.33 ml/kg. Blood samples were collected at the times indicated in Tables 17-18 and were analyzed according to General Procedure B. Results of the PK experiments are described in Tables 17-18 and FIGS. 17-18.

TABLE 17
Plasma concentration vs. time data for a study evaluating Formulation
H administered to rats subcutaneously at 0.33 mL/kg (n = 3).

| | | | | | | Std. Dev. |
|---|---|---|---|---|---|---|
| | | Rat 1 | Rat 2 | Rat 3 | Avg. Conc. | Conc. |
| Days | Hours | (ng/mL) | (ng/mL) | (ng/mL) | (ng/mL) | (ng/mL) |
| 0.0 | 1.0 | 13.5 | 4.0 | 4.8 | 7.4 | 5.3 |
| 0.2 | 4.0 | 46.7 | 17.7 | 24.0 | 29.5 | 15.3 |
| 0.3 | 7.0 | 70.3 | 35.9 | 46.8 | 51.0 | 17.6 |
| 1 | 24 | 113.0 | 75.2 | 70.9 | 86.4 | 23.2 |
| 2 | 48 | 113.0 | 72.6 | 83.4 | 89.7 | 20.9 |
| 4 | 96 | 70.2 | 58.5 | 62.9 | 63.9 | 5.9 |
| 6 | 144 | 69.7 | 41.5 | 70.4 | 60.5 | 16.5 |
| 13 | 312 | 81.1 | 52.8 | 125.0 | 86.3 | 36.4 |
| 20 | 480 | 89.3 | 60.9 | 162.0 | 104.1 | 52.1 |
| 27 | 648 | 90.9 | 80.8 | 241.0 | 137.6 | 89.7 |
| 34 | 816 | 115.0 | 96.2 | 211.0 | 140.7 | 61.6 |
| 41 | 984 | 171.0 | 111.0 | 227.0 | 169.7 | 58.0 |
| 48 | 1152 | 176.0 | 98.6 | 201.0 | 158.5 | 53.4 |
| 55 | 1320 | 176.0 | 130.0 | 233.0 | 179.7 | 51.6 |
| 62 | 1488 | 145.0 | 130.0 | 210.0 | 161.7 | 42.5 |
| 69 | 1656 | 108.0 | 131.0 | 159.0 | 132.7 | 25.5 |
| 76 | 1824 | 138.0 | 102.0 | 121.0 | 120.3 | 18.0 |
| 83 | 1992 | 141.0 | 125.0 | 120.0 | 128.7 | 11.0 |
| 90 | 2160 | 87.9 | 110.0 | 104.0 | 100.6 | 11.4 |
| 97 | 2328 | 116.0 | 147.0 | 105.0 | 122.7 | 21.8 |
| 104 | 2496 | 79.5 | 92.6 | 67.8 | 80.0 | 12.4 |

LOD = Limit of detection

TABLE 18
Plasma concentration vs. time data for a study evaluating Formulation
H administered to rats intramuscularly at 0.33 mL/kg (n = 3).

| | | | | | | Std. Dev. |
|---|---|---|---|---|---|---|
| | | Rat 1 | Rat 2 | Rat 3 | Avg. Conc. | Conc. |
| Days | Hours | (ng/mL) | (ng/mL) | (ng/mL) | (ng/mL) | (ng/mL) |
| 0.0 | 1.0 | 60.0 | 44.9 | 36.2 | 47.0 | 12.0 |
| 0.2 | 4.0 | 187.0 | 185.0 | 130.0 | 167.3 | 32.3 |
| 0.3 | 7.0 | 340.0 | 312.0 | 206.0 | 286.0 | 70.7 |
| 1 | 24 | 456.0 | 441.0 | 411.0 | 436.0 | 22.9 |
| 2 | 48 | 305.0 | 341.0 | 286.0 | 310.7 | 27.9 |
| 4 | 96 | 295.0 | 437.0 | 268.0 | 333.3 | 90.8 |
| 6 | 144 | 482.0 | 676.0 | 528.0 | 562.0 | 101.4 |
| 13 | 312 | 344.0 | 415.0 | 392.0 | 383.7 | 36.2 |
| 20 | 480 | 315.0 | 454.0 | 448.0 | 405.7 | 78.6 |
| 27 | 648 | 185.0 | 297.0 | 348.0 | 276.7 | 83.4 |
| 34 | 816 | 119.0 | 183.0 | 273.0 | 191.7 | 77.4 |
| 41 | 984 | 102.0 | 186.0 | 212.0 | 166.7 | 57.5 |
| 48 | 1152 | 65.1 | 131.0 | 156.0 | 117.4 | 47.0 |
| 55 | 1320 | 42.5 | 113.0 | 121.0 | 92.2 | 43.2 |
| 62 | 1488 | 27.6 | 79.7 | 98.0 | 68.4 | 36.5 |
| 69 | 1656 | 16.5 | 49.6 | 59.5 | 41.9 | 22.5 |
| 76 | 1824 | 11.9 | 53.4 | 48.9 | 38.1 | 22.8 |
| 83 | 1992 | 8.2 | 47.4 | 41.6 | 32.4 | 21.2 |
| 90 | 2160 | 4.6 | 44.5 | 33.7 | 27.6 | 20.7 |
| 97 | 2328 | 3.9 | 33.2 | 33.3 | 23.5 | 16.9 |

LOD = Limit of detection

### Procedure for Measuring Pharmacokinetic Parameters for Formulation I in an In Vivo Experiment

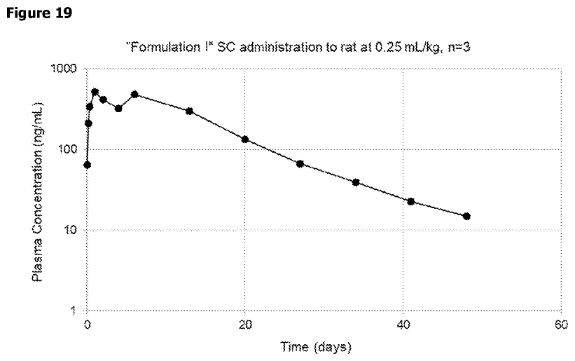

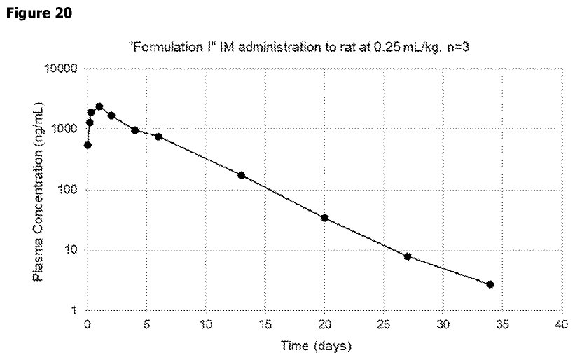

“Formulation I” was administered to Wistar Han Rats as either a subcutaneous injection at a dose of 0.25 ml/kg or as an intramuscular injection at a dose of 0.25 ml/kg. Blood samples were collected at the times indicated in Tables 19-20 and were analyzed according to General Procedure A. Results of the PK experiments are described in Tables 19-20 and FIGS. 19-20.

TABLE 19
Plasma concentration vs. time data for a study evaluating Formulation
I administered to rats subcutaneously at 0.25 mL/kg (n = 3).

| | | | | | | Std. Dev. |
|---|---|---|---|---|---|---|
| | | Rat 1 | Rat 2 | Rat 3 | Avg. Conc. | Conc. |
| Days | Hours | (ng/mL) | (ng/mL) | (ng/mL) | (ng/mL) | (ng/mL) |
| 0.04 | 1.0 | 79.2 | 64.9 | 49.6 | 64.6 | 14.8 |
| 0.17 | 4.0 | 253.0 | 217.0 | 159.0 | 209.7 | 47.4 |
| 0.29 | 7.0 | 302.0 | 426.0 | 284.0 | 337.3 | 77.3 |
| 1 | 24 | 465.0 | 580.0 | 504.0 | 516.3 | 58.5 |
| 2 | 48 | 344.0 | 427.0 | 475.0 | 415.3 | 66.3 |
| 4 | 96 | 257.0 | 340.0 | 370.0 | 322.3 | 58.5 |
| 6 | 144 | 360.0 | 437.0 | 647.0 | 481.3 | 148.5 |
| 13 | 312 | 212.0 | 352.0 | 338.0 | 300.7 | 77.1 |
| 20 | 480 | 118.0 | 109.0 | 173.0 | 133.3 | 34.6 |
| 27 | 648 | 72.9 | 49.9 | 78.1 | 67.0 | 15.0 |
| 34 | 816 | 43.7 | 24.8 | 49.4 | 39.3 | 12.9 |
| 41 | 984 | 24.7 | 14.9 | 27.9 | 22.5 | 6.8 |
| 48 | 1152 | 22.5 | 10.2 | 12.1 | 14.9 | 6.6 |

LOD = Limit of detection

TABLE 20
Plasma concentration vs. time data for a study evaluating Formulation
I administered to rats intramuscularly at 0.25 mL/kg (n = 3).

| | | | | | | Std. Dev. |
|---|---|---|---|---|---|---|
| | | Rat 1 | Rat 2 | Rat 3 | Avg. Conc. | Conc. |
| Days | Hours | (ng/mL) | (ng/mL) | (ng/mL) | (ng/mL) | (ng/mL) |
| 0.0 | 1.0 | 694.0 | 468.0 | 462.0 | 541.3 | 132.2 |
| 0.2 | 4.0 | 1480.0 | 1330.0 | 1020.0 | 1276.7 | 234.6 |
| 0.3 | 7.0 | 2100.0 | 1940.0 | 1580.0 | 1873.3 | 266.3 |
| 1 | 24 | 2490.0 | 2680.0 | 1950.0 | 2373.3 | 378.7 |
| 2 | 48 | 1650.0 | 2040.0 | 1300.0 | 1663.3 | 370.2 |
| 4 | 96 | 794.0 | 1120.0 | 947.0 | 953.7 | 163.1 |
| 6 | 144 | 679.0 | 768.0 | 812.0 | 753.0 | 67.8 |
| 13 | 312 | 142.0 | 212.0 | 167.0 | 173.7 | 35.5 |
| 20 | 480 | 10.4 | 41.0 | 51.2 | 34.2 | 21.2 |
| 27 | 648 | <LOD | 4.7 | 10.9 | 7.8 | |
| 34 | 816 | <LOD | 1.2 | 4.2 | 2.7 | |

LOD = Limit of detection

### Procedure for Measuring Pharmacokinetic Parameters for Formulation J in an In Vivo Experiment

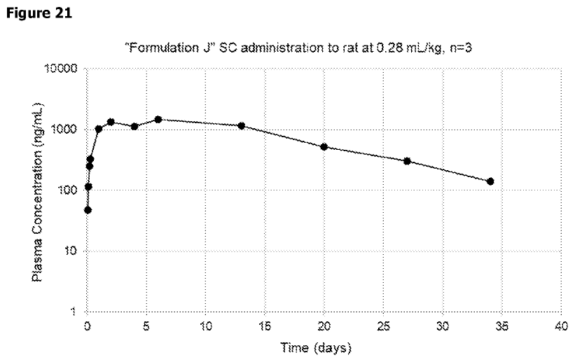

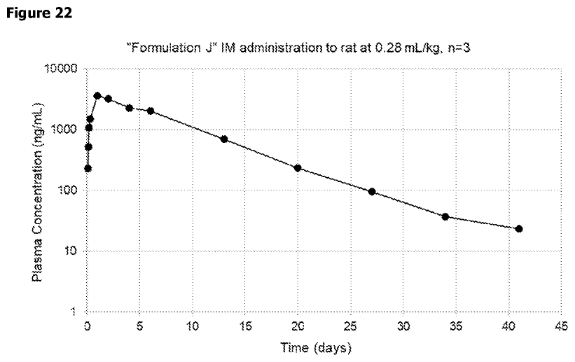

“Formulation J” was administered to Wistar Han Rats as either a subcutaneous injection at a dose of 0.28 mL/kg or as an intramuscular injection at a dose of 0.28 ml/kg. Blood samples were collected at the times indicated in Tables 21-22 and were analyzed according to General Procedure A. Results of the PK experiments are described in Tables 21-22 and FIGS. 21-22.

TABLE 21
Plasma concentration vs. time data for a study evaluating Formulation
J administered to rats subcutaneously at 0.28 mL/kg (n = 3).

| | | | | | | Std. Dev. |
|---|---|---|---|---|---|---|
| | | Rat 1 | Rat 2 | Rat 3 | Avg. Conc. | Conc. |
| Days | Hours | (ng/mL) | (ng/mL) | (ng/mL) | (ng/mL) | (ng/mL) |
| 0.04 | 1 | 17.2 | 12.8 | 114 | 48.0 | 57.2 |
| 0.13 | 3 | 57 | 59.4 | 232 | 116.1 | 100.4 |
| 0.21 | 5 | 116 | 134 | 507 | 252.3 | 220.7 |
| 0.29 | 7 | 182 | 198 | 611 | 330.3 | 243.2 |
| 1 | 24 | 850 | 730 | 1520 | 1033.3 | 425.7 |
| 2 | 48 | 1400 | 1270 | 1340 | 1336.7 | 65.1 |
| 4 | 96 | 1240 | 1190 | 1020 | 1150.0 | 115.3 |
| 6 | 144 | 1520 | 1760 | 1230 | 1503.3 | 265.4 |
| 13 | 312 | 1230 | 1560 | 725 | 1171.7 | 420.5 |
| 20 | 480 | 492 | 823 | 261 | 525.3 | 282.5 |
| 27 | 648 | 250 | 545 | 134 | 309.7 | 211.9 |
| 34 | 816 | 124 | 250 | 50.8 | 141.6 | 100.8 |
| 41 | 984 | 62.5 | 103 | 19.6 | 61.7 | 41.7 |

LOD = Limit of detection

TABLE 22
Plasma concentration vs. time data for a study evaluating Formulation
J administered to rats intramuscularly at 0.28 mL/kg (n = 3).

| | | | | | | Std. Dev. |
|---|---|---|---|---|---|---|
| | | Rat 1 | Rat 2 | Rat 3 | Avg. Conc. | Conc. |
| Days | Hours | (ng/mL) | (ng/mL) | (ng/mL) | (ng/mL) | (ng/mL) |
| 0.04 | 1 | 190 | 237 | 262 | 229.7 | 36.6 |
| 0.13 | 3 | 405 | 503 | 676 | 528.0 | 137.2 |
| 0.21 | 5 | 830 | 1110 | 1310 | 1083.3 | 241.1 |
| 0.29 | 7 | 1100 | 1470 | 1970 | 1513.3 | 436.6 |
| 1 | 24 | 2670 | 4650 | 3650 | 3656.7 | 990.0 |
| 2 | 48 | 2710 | 3850 | 3060 | 3206.7 | 584.0 |
| 4 | 96 | 2760 | 2400 | 1670 | 2276.7 | 555.4 |
| 6 | 144 | 3300 | 2090 | 753 | 2047.7 | 1274.0 |
| 13 | 312 | 1050 | 803 | 231 | 694.7 | 420.1 |
| 20 | 480 | 387 | 274 | 40.1 | 233.7 | 176.9 |
| 27 | 648 | 135 | 55.6 | | 95.3 | 56.1 |
| 34 | 816 | 63.5 | 11.5 | | 37.5 | 36.8 |
| 41 | 984 | 23.7 | | | 23.7 | |

LOD = Limit of detection

The data generated above and depicted in the Figures shows that the pharmaceutical compositions of the invention extend the release profile of the compounds of Formula Ia and Formula Ib and suggest their use in long-acting administration of the compounds.

What is claimed is:

1. A pharmaceutical composition comprising a compound of Formula Ia, or a pharmaceutically acceptable salt thereof, 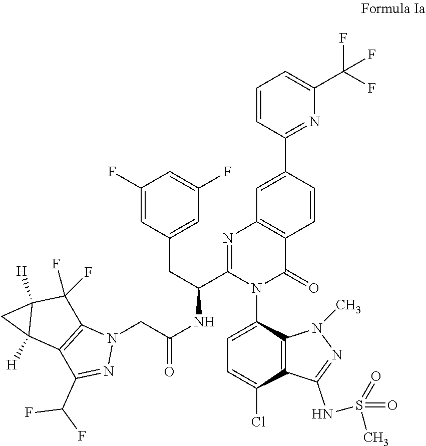
wherein the composition comprises polyethylene glycol and ethanol.

2. A pharmaceutical composition comprising a compound of Formula Ib, or a pharmaceutically acceptable salt thereof, 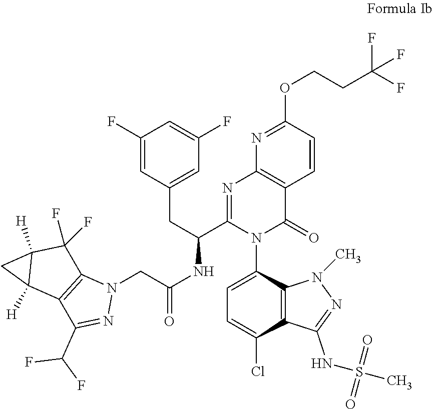
wherein the composition comprises polyethylene glycol and ethanol.

3. The pharmaceutical composition according to claim 1 wherein the composition further comprises one or more of water, lecithin, propylene glycol, benzyl alcohol, or sesame oil.

4. The pharmaceutical composition according to claim 3 wherein the composition comprises lecithin.

5. The pharmaceutical composition according to claim 4 wherein the lecithin is egg-based or soy-based and is about 80 weight % phosphatidylcholine.

6. The pharmaceutical composition according to claim 4 wherein the lecithin is egg-based or soy-based and is about 100 weight % phosphatidylcholine.

7. The pharmaceutical composition according to claim 4 further comprising propylene glycol, benzyl alcohol, or sesame oil.

8. The pharmaceutical composition according to claim 1 wherein the average molecular weight of polyethylene glycol is about 200 (PEG 200).

9. The pharmaceutical composition according to claim 1 wherein the average molecular weight of polyethylene glycol is about 300 (PEG 300).

10. The pharmaceutical composition according to claim 1 wherein the average molecular weight of polyethylene glycol is about 400 (PEG 400).

11. The pharmaceutical composition according to claim 1 wherein the amount of ethanol is about 5-25 weight %.

12. The pharmaceutical composition according to claim 1 wherein the amount of ethanol is about 20 weight %.

13. The pharmaceutical composition according to claim 1 wherein the amount of polyethylene glycol is about 40-50% by weight.

14. The pharmaceutical composition according to claim 1 wherein the composition is a homogeneous solution.

15. The pharmaceutical composition according to claim 1 comprising about 20% by weight of the compound of Formula Ia, or a pharmaceutically acceptable salt thereof, about 45% by weight of PEG200, about 20% by weight of ethanol, and about 15% by weight of lecithin.

16. The pharmaceutical composition according to claim 1 comprised of about 20% by weight of the compound of Formula Ib, or a pharmaceutically acceptable salt thereof, about 45% by weight of PEG200, about 20% by weight of ethanol, and about 15% by weight of lecithin.

17. A pharmaceutical composition comprising a compound of Formula Ia, 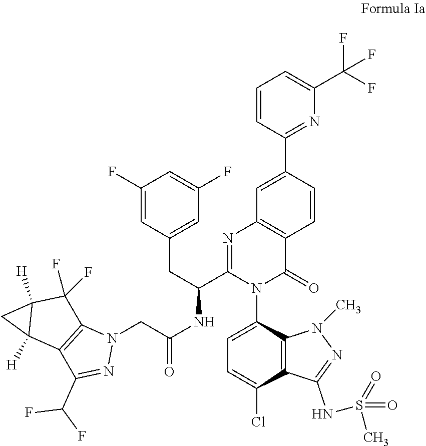
wherein the composition comprises polyethylene glycol and ethanol.

18. A pharmaceutical composition comprising a compound of Formula Ib, 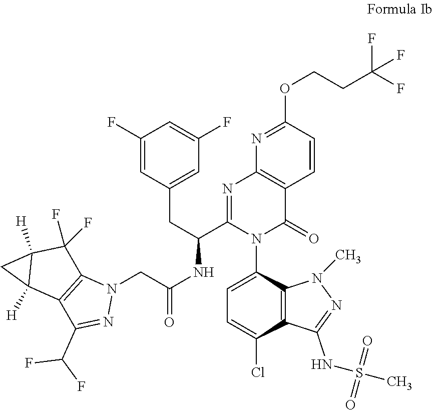
wherein the composition comprises polyethylene glycol and ethanol.

19. The pharmaceutical composition according to claim 17 wherein the composition further comprises one or more of water, lecithin, propylene glycol, benzyl alcohol, or sesame oil.

20. The pharmaceutical composition according to claim 19 wherein the composition comprises lecithin.

21. The pharmaceutical composition according to claim 20 wherein the lecithin is egg-based or soy-based and is about 80 weight % phosphatidylcholine.

22. The pharmaceutical composition according to claim 20 wherein the lecithin is egg-based or soy-based and is about 100 weight % phosphatidylcholine.

23. The pharmaceutical composition according to claim 20 further comprising propylene glycol, benzyl alcohol, or sesame oil.

24. The pharmaceutical composition according to claim 17 wherein the average molecular weight of polyethylene glycol is about 200 (PEG 200).

25. The pharmaceutical composition according to claim 17 wherein the average molecular weight of polyethylene glycol is about 300 (PEG 300).

26. The pharmaceutical composition according to claim 17 wherein the average molecular weight of polyethylene glycol is about 400 (PEG 400).

27. The pharmaceutical composition according to claim 17 wherein the amount of ethanol is about 5-25 weight %.

28. The pharmaceutical composition according to claim 17 wherein the amount of ethanol is about 20 weight %.

29. The pharmaceutical composition according to claim 17 wherein the amount of polyethylene glycol is about 40-50% by weight.

30. The pharmaceutical composition according to claim 17 wherein the composition is a homogeneous solution.

31. The pharmaceutical composition according to claim 17 comprised of about 20% by weight of the compound of Formula Ia, about 45% by weight of PEG200, about 20% by weight of ethanol, and about 15% by weight of lecithin.

32. The pharmaceutical composition according to claim 18 comprised of about 20% by weight of the compound of Formula Ib, about 45% by weight of PEG200, about 20% by weight of ethanol, and about 15% by weight of lecithin.

33. A pharmaceutical composition comprising a compound of Formula Ia, or a pharmaceutically acceptable salt thereof, 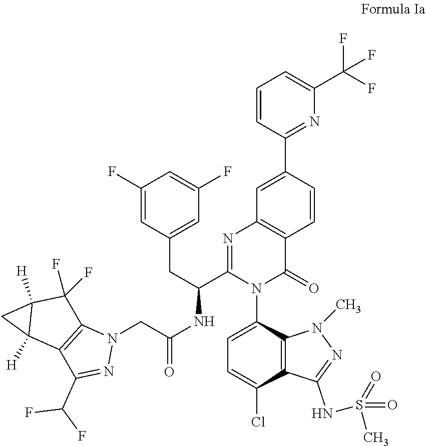
wherein the composition comprises water and contains less than 1% by weight of polyethylene glycol.

34. A pharmaceutical composition comprising a compound of Formula Ib, or a pharmaceutically acceptable salt thereof, 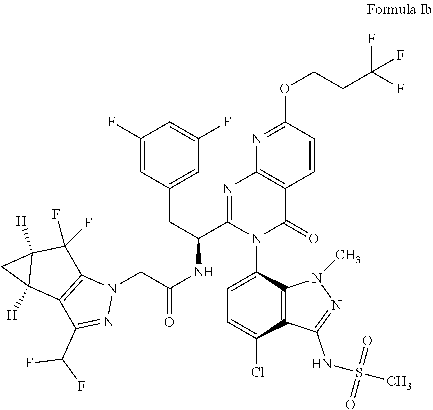
wherein the composition comprises water and contains less than 1% by weight of polyethylene glycol.

35. The pharmaceutical composition according to claim 33 further comprising one or more of sodium acetate, acetic acid, mannitol, sodium chloride, Poloxamer 338, or Poloxamer 188.

36. The pharmaceutical composition according to claim 35 wherein the composition comprises Poloxamer 338 or Poloxamer 188.

37. The pharmaceutical composition according claim 36 wherein the composition further comprises sodium acetate and acetic acid.

38. The pharmaceutical composition according to claim 37 wherein the composition further comprises mannitol or sodium chloride.

39. The pharmaceutical composition according to claim 33 wherein the mean particle diameter of the compound of Formula Ia is 0.2 μm to 0.5 μm.

40. The pharmaceutical composition according to claim 33 wherein the mean particle diameter of the compound of Formula Ia is ≤0.2 μm.

41. The pharmaceutical composition according to claim 34 wherein the mean particle diameter of the compound of Formula Ib is 0.2 μm to 0.5 μm.

42. The pharmaceutical composition according to claim 34 wherein the mean particle diameter of the compound of Formula Ib is ≤0.2 μm.

43. The pharmaceutical composition according to claim 33 comprising about 300 mg/mL of the compound of Formula Ia, or a pharmaceutically acceptable salt thereof, about 5.4% by weight of P338, about 3.5% by weight of mannitol, and the remainder of the composition as water or aqueous acetate buffer.

44. The pharmaceutical composition according to claim 34 comprising about 300 mg/mL of the compound of Formula Ib, or a pharmaceutically acceptable salt thereof, about 5.4% by weight of P338, about 3.5% by weight of mannitol, and the remainder of the composition as water or aqueous acetate buffer.

45. The pharmaceutical composition according to claim 33 which is a heterogeneous suspension.

46. A pharmaceutical composition comprising a compound of Formula Ia, 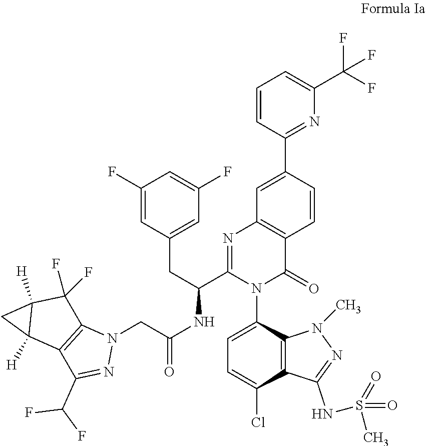
wherein the composition comprises water and contains less than 1% by weight of polyethylene glycol.

47. A pharmaceutical composition comprising a compound of Formula Ib, 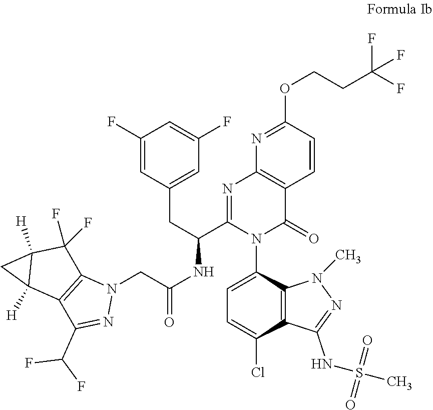
wherein the composition comprises water and contains less than 1% by weight of polyethylene glycol.

48. The pharmaceutical composition according to claim 46 further comprising one or more of sodium acetate, acetic acid, mannitol, sodium chloride, Poloxamer 338, or Poloxamer 188.

49. The pharmaceutical composition according to claim 48 wherein the composition comprises Poloxamer 338 or Poloxamer 188.

50. The pharmaceutical composition according claim 49 wherein the composition further comprises sodium acetate and acetic acid.

51. The pharmaceutical composition according to claim 50 wherein the composition further comprises mannitol or sodium chloride.

52. The pharmaceutical composition according to claim 46 wherein the mean particle diameter of the compound of Formula Ia is 0.2 μm to 0.5 μm.

53. The pharmaceutical composition according to claim 46 wherein the mean particle diameter of the compound of Formula Ia is ≤0.2 μm.

54. The pharmaceutical composition according to claim 47 wherein the mean particle diameter of the compound of Formula Ib is 0.2 μm to 0.5 μm.

55. The pharmaceutical composition according to claim 47 wherein the mean particle diameter of the compound of Formula Ib is ≤0.2 μm.

56. The pharmaceutical composition according to claim 46 comprising about 300 mg/mL of the compound of Formula Ia, about 5.4% by weight of P338, about 3.5% by weight of mannitol, and the remainder of the composition as water or aqueous acetate buffer.

57. The pharmaceutical composition according to claim 47 comprising about 300 mg/mL of the compound of Formula Ib, about 5.4% by weight of P338, about 3.5% by weight of mannitol, and the remainder of the composition as water or aqueous acetate buffer.

58. The pharmaceutical composition according to claim 46 which is a heterogeneous suspension.

59. A method of treating HIV infection in a human comprising administration of a therapeutically effective amount of a pharmaceutical composition according to claim 1.

60. The method according to claim 59 wherein said administration is via intramuscular injection.

61. The method according to claim 59 wherein said administration is via subcutaneous injection.

62. The method according to claim 59 wherein said method further comprises administration of at least one other agent used for treating HIV infection in a human.

63. The method according to claim 62 wherein the at least one other agent is selected from the group consisting of abacavir, atazanavir, bictegravir, cabotegravir, dolutegravir, fostemsavir, lamivudine, maraviroc, rilpiverine, tenofovir disoproxil, tenofovir, tenofovir afenamide, islatravir, doravirine, preziata, S-648414, GSK3640254, N6LS, GSK3739937/VH3739937, GSK4000422/VH4000422, GSK4023991/VH4023991 and S-365598.

64. The method according to claim 62 wherein the at least one other agent is selected from the group consisting of dolutegravir, lamivudine, fostemsavir, cabotegravir, N6LS, GSK3739937/VH3739937, GSK4000422/VH4000422, GSK4023991/VH4023991 and S-365598.

65. The method according to claim 62 wherein the at least one other agent is selected from the group consisting of dolutegravir, bictegravir, islatravir, lamivudine, fostemsavir, and cabotegravir.

66-68. (canceled)
